(19) United States
(12) Patent Application Publication
(10) Pub. No.: US 2025/0231036 A1
(43) Pub. Date: Jul. 17, 2025

(54) AUTONOMOUS DRIVING VEHICLE, CONTROL DEVICE, CONTROL METHOD, AND STORAGE MEDIUM STORING CONTROL PROGRAM

(71) Applicant: Mitsubishi Electric Corporation, TOKYO (JP)

(72) Inventors: Kotoyu SASAYAMA, Tokyo (JP); Tomoya UEFUNE, Tokyo (JP); Michinori YOSHIDA, Tokyo (JP); Munetaka NISHIHIRA, Tokyo (JP)

(73) Assignee: Mitsubishi Electric Corporation, TOKYO (JP)

(21) Appl. No.: 18/984,102

(22) Filed: Dec. 17, 2024

Related U.S. Application Data
(63) Continuation of application No. PCT/JP2022/028220, filed on Jul. 20, 2022.

(51) Int. Cl. G01C 21/34; A61G 5/04; A61M 21/00; A61M 21/02; B62J 3/00; B62J 50/22; G05D 1/617; G05D 105/22

(52) U.S. Cl. CPC G01C 21/3484; A61M 21/02; B62J 50/22; G05D 1/618; A61G 5/04; A61G 2203/22; A61M 2021/0022; A61M 2021/0027; A61M 2021/0044; A61M 2021/005; A61M 2205/3303; B62J 3/00; G05D 2105/24

(57) ABSTRACT

An autonomous driving vehicle includes a vicinity sensor, an information presentation unit, an operation unit, a collection unit to previously collect an anxiety start position based on the experiment participant's operation on the operation unit along a route, and a control unit to control operation of the autonomous driving vehicle based on road information, vehicle information, vicinity detection information, and the anxiety start position. The control unit sets a presentation start position, as a position where the information presentation unit is made to start presenting anxiety reduction information, before the anxiety start position in the route of autonomous traveling with a user riding therein, and makes the information presentation unit start the presentation of the anxiety reduction information when the autonomous driving vehicle with the user riding therein reaches the presentation start position.

### CROSS-REFERENCE TO RELATED APPLICATION

This application is a continuation application of International Application No. PCT/JP2022/028220 having an international filing date of Jul. 20, 2022.

### BACKGROUND OF THE INVENTION

### 1. Field of the Invention

The present disclosure relates to an autonomous driving vehicle, a control device of an autonomous driving vehicle, a control method of an autonomous driving vehicle, and a control program of an autonomous driving vehicle.

### 2. Description of the Related Art

There has been proposed an information presentation method of presenting relief information for relieving stress to a user riding in a mobile object (see Patent Reference 1, for example). In this information presentation method, the stress relief information is presented in a presentation mode determined based on a physiological index (i.e., biological information) previously acquired from a subject during autonomous traveling of the mobile object.

Patent Reference 1: Japanese Patent Application Publication No. 2016-052374 (see abstract, for example).

However, the above-described conventional information presentation method is not designed to handle a situation that hardly changes the physiological index. Therefore, anxiety relief information (i.e., anxiety reduction information) cannot be presented at a time point when the physiological index is hardly changed, such as when the user is at a position far from an anxiety factor situated in front. Further, the relief information cannot be presented in cases where a change in the behavior of the mobile object (e.g., acceleration in a front-back direction, a left-right direction or an up-down direction) does not occur when the mobile object passes through the position of the anxiety factor.

### SUMMARY OF THE INVENTION

An object of the present disclosure is to provide an autonomous driving vehicle and a control device, a control method and a control program of an autonomous driving vehicle capable of presenting the anxiety reduction information at an appropriate position in the autonomous traveling.

An autonomous driving vehicle in the present disclosure is a vehicle that travels autonomously with a user riding therein, including a vicinity sensor to detect at least a situation in front in a traveling direction; an information presentation device to present information to the user; an operation device; and processing circuitry to previously collect an anxiety start position, as a position before an anxiety factor that makes an experiment participant feel anxiety in a route of autonomous traveling of the autonomous driving vehicle with the experiment participant riding therein and a position where the experiment participant starts feeling anxiety about the anxiety factor, based on the experiment participant's operation on the operation device along the route; and to control operation of the autonomous driving vehicle based on road information including a map of a scheduled route to a destination of the autonomous driving vehicle and a road attribute of the scheduled route, vehicle information regarding the autonomous driving vehicle, vicinity detection information outputted from the vicinity sensor, and the anxiety start position. The processing circuitry sets a presentation start position, as a position where the information presentation device is made to start presenting anxiety reduction information, before the anxiety start position in the route of the autonomous traveling with the user riding therein, and makes the information presentation device start the presentation of the anxiety reduction information when the autonomous driving vehicle with the user riding therein reaches the presentation start position.

A control method of an autonomous driving vehicle in the present disclosure is a control method of an autonomous driving vehicle that includes a vicinity sensor to detect at least a situation in front in a traveling direction, an information presentation device to present information to a user, and an operation device. The control method includes previously collecting an anxiety start position, as a position before an anxiety factor that makes an experiment participant feel anxiety in a route of autonomous traveling of the autonomous driving vehicle with the experiment participant riding therein and a position where the experiment participant starts feeling anxiety about the anxiety factor, based on the experiment participant's operation on the operation device along the route; and controlling operation of the autonomous driving vehicle based on road information including a map of a scheduled route to a destination of the autonomous driving vehicle and a road attribute of the scheduled route, vehicle information regarding the autonomous driving vehicle, vicinity detection information outputted from the vicinity sensor, and the anxiety start position, wherein in the controlling the operation of the autonomous driving vehicle, a presentation start position as a position where the information presentation device is made to start presenting anxiety reduction information is set before the anxiety start position in the route of the autonomous traveling with the user riding therein, and the information presentation device is made to start the presentation of the anxiety reduction information when the autonomous driving vehicle with the user riding therein reaches the presentation start position.

According to the present disclosure, the anxiety of the user can be reduced sufficiently by presenting the anxiety reduction information at an appropriate position in the autonomous traveling.

### BRIEF DESCRIPTION OF THE DRAWINGS

Further scope of applicability of the present invention will become apparent from the detailed description given hereinafter. However, it should be understood that the detailed description and specific examples, while indicating preferred embodiments of the invention, are given by way of illustration only, since various changes and modifications will become apparent to those skilled in the art from the detailed description,

FIG. 1 is a block diagram schematically showing the configuration of an autonomous driving vehicle according to a first embodiment;

FIG. 2 is a schematic diagram showing an autonomous driving wheelchair as the autonomous driving vehicle in autonomous traveling;

FIG. 3 is a schematic diagram showing a case where the autonomous driving vehicle in FIG. 2 is passing through a recognition start position of an anxiety factor;

FIG. 4 is a schematic diagram showing the autonomous driving vehicle traveling towards a destination by passing through the recognition start position of the anxiety factor, a presentation start position of anxiety reduction information, an anxiety start position, and a passage start position and a passage end position of the anxiety factor;

FIG. 5 is a diagram showing an example of the hardware configuration of a control device of the autonomous driving vehicle according to the first embodiment;

FIG. 6 is a schematic plan view showing an example in which the autonomous driving vehicle travels on a scheduled route even though there exists a narrow road width as the anxiety factor;

FIG. 7 is a schematic plan view showing an example in which there exists a narrow road width as the anxiety factor and the autonomous driving vehicle travels on a detour route;

FIG. 8 is a diagram showing an example 1 of the anxiety reduction information presented by an information presentation unit of the autonomous driving vehicle;

FIG. 9 is a diagram showing an example 2 of the anxiety reduction information presented by the information presentation unit of the autonomous driving vehicle;

FIG. 10 is a diagram showing an example 3 of the anxiety reduction information presented by the information presentation unit of the autonomous driving vehicle;

FIG. 11 is a diagram showing an example 4 of the anxiety reduction information presented by the information presentation unit of the autonomous driving vehicle;

FIG. 12 is a diagram showing a detour route selection screen presented by the information presentation unit of the autonomous driving vehicle;

FIG. 13 is a diagram showing an example 5 of the anxiety reduction information presented by the information presentation unit of the autonomous driving vehicle;

FIG. 14 is a flowchart showing the operation of the control device of the autonomous driving vehicle according to the first embodiment;

FIG. 15 is a schematic diagram showing an example in which an autonomous driving vehicle according to a second embodiment travels on the scheduled route even though there exists a slope as an anxiety factor;

FIG. 16 is a schematic diagram showing an example in which there exists a slope as the anxiety factor and the autonomous driving vehicle according to the second embodiment avoids the scheduled route;

FIG. 17 is a diagram showing an example 6 of the anxiety reduction information presented by the information presentation unit of the autonomous driving vehicle;

FIG. 18 is a flowchart showing the operation of the control device of the autonomous driving vehicle according to the second embodiment;

FIG. 19 is a schematic diagram showing an example in which an autonomous driving vehicle according to a third embodiment travels on the scheduled route even though there exists a step as an anxiety factor;

FIG. 20 is a schematic diagram showing an example in which there exists a step as the anxiety factor and the autonomous driving vehicle according to the third embodiment avoids the scheduled route;

FIG. 21 is a diagram showing an example 7 of the anxiety reduction information presented by the information presentation unit of the autonomous driving vehicle;

FIG. 22 is a flowchart showing the operation of the control device of the autonomous driving vehicle according to the third embodiment;

FIG. 23 is a schematic diagram showing an example in which a user riding in an autonomous driving vehicle according to a modification of the third embodiment has recognized unevenness;

FIG. 24 is a schematic plan view showing the operation of an autonomous driving vehicle according to a fourth embodiment when a mobile object as an anxiety factor approaches the autonomous driving vehicle;

FIG. 25 is a schematic plan view showing the operation of the autonomous driving vehicle according to the fourth embodiment when the mobile object as the anxiety factor approaches the autonomous driving vehicle;

FIG. 26 is a diagram showing an example 8 of the anxiety reduction information presented by the information presentation unit of the autonomous driving vehicle;

FIG. 27 is a flowchart showing the operation of the control device of the autonomous driving vehicle according to the fourth embodiment; and

FIG. 28 is a diagram showing an example 9 of the anxiety reduction information presented by the information presentation unit of the autonomous driving vehicle.

### DETAILED DESCRIPTION OF THE INVENTION

An autonomous driving vehicle, a control device of an autonomous driving vehicle, a control method of an autonomous driving vehicle, and a control program of an autonomous driving vehicle according to each embodiment will be described below with reference to the drawings. The following embodiments are just examples and it is possible to appropriately combine embodiments and appropriately modify each embodiment.

### First Embodiment

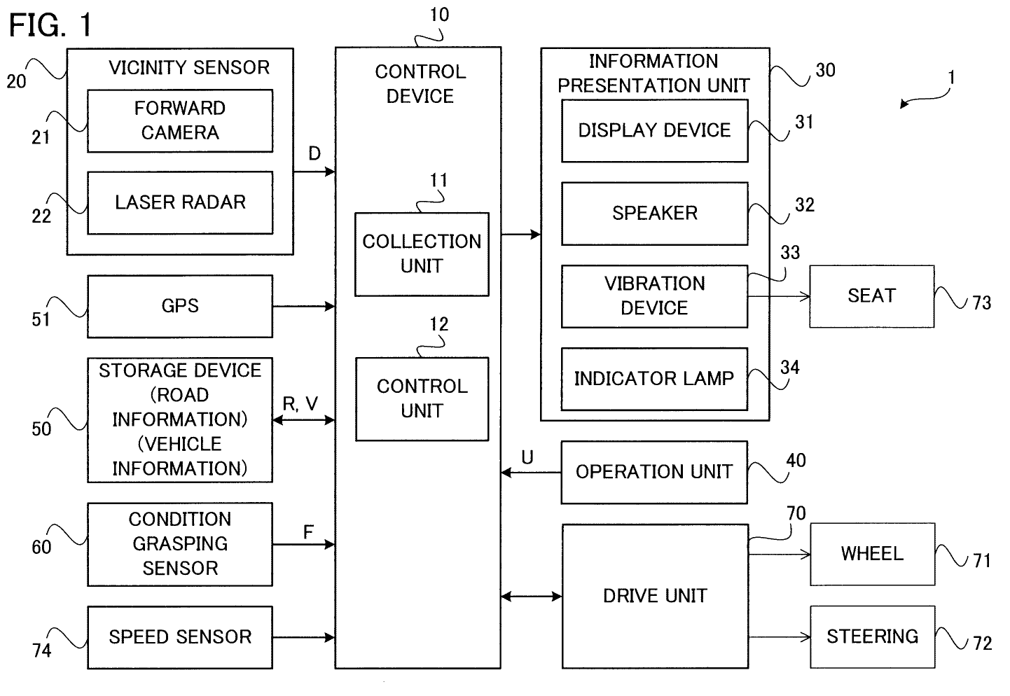

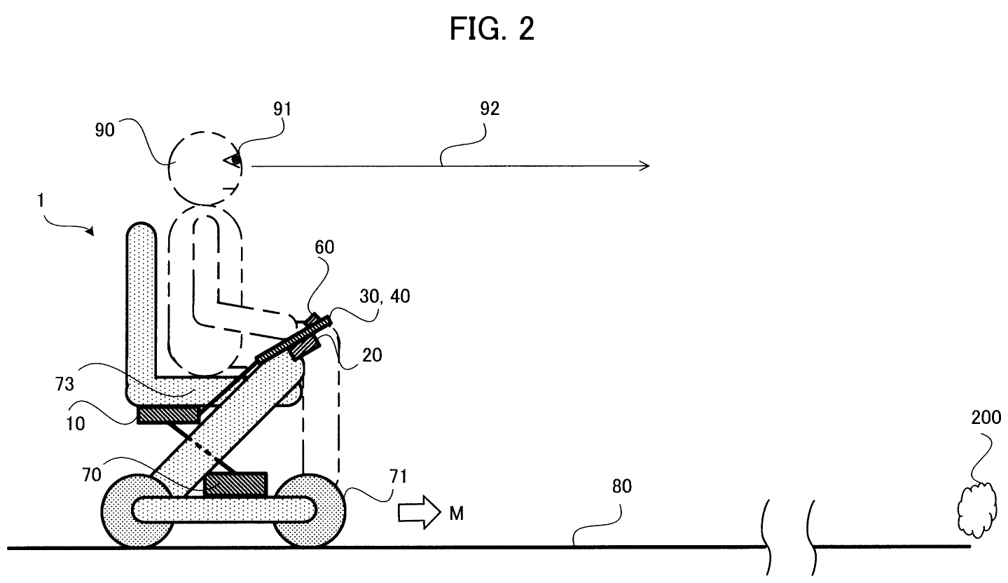

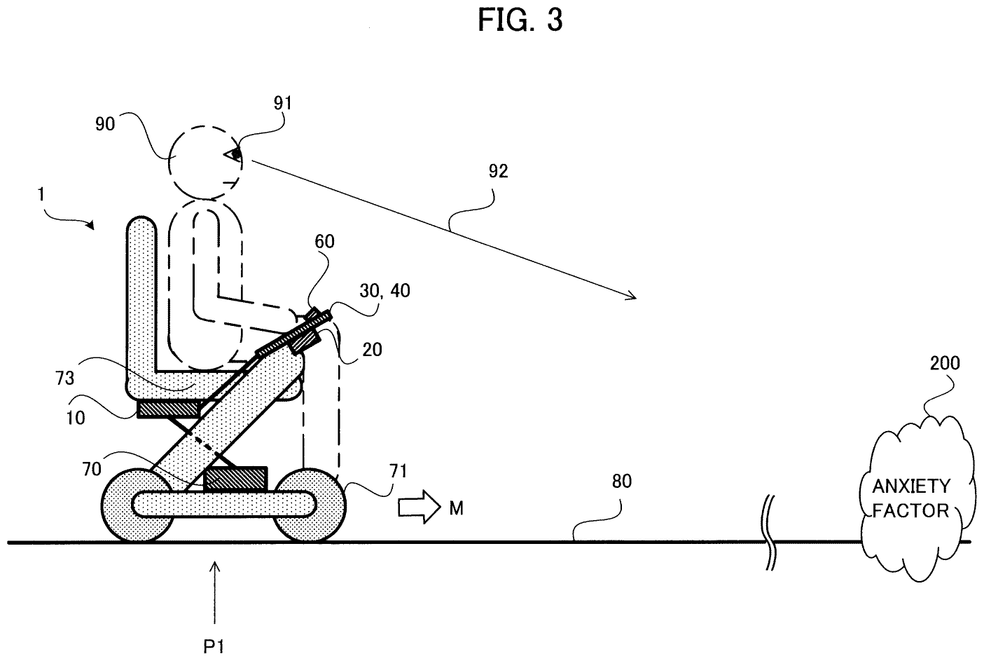

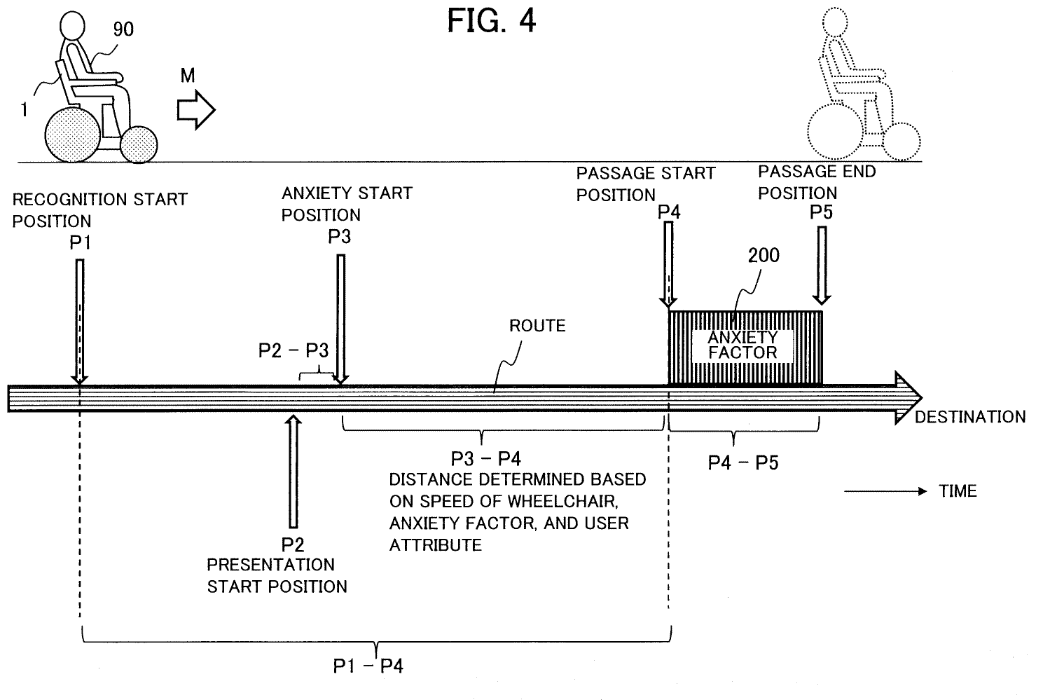

FIG. 1 is a block diagram schematically showing the configuration of an autonomous driving vehicle 1 according to a first embodiment. FIG. 2 is a schematic diagram showing an autonomous driving wheelchair as the autonomous driving vehicle 1 in autonomous traveling, and FIG. 3 is a schematic diagram showing a case where the autonomous driving vehicle 1 is passing through a recognition start position P1 of an anxiety factor 200. FIG. 4 is a schematic diagram showing the autonomous driving vehicle 1 traveling towards a destination (e.g., goal) by passing through the recognition start position P1 of the anxiety factor 200, a presentation start position P2 of the anxiety reduction information, an anxiety start position P3 as a position where anxiety starts to be felt, and a passage start position P4 and a passage end position P5 of the anxiety factor 200. The autonomous driving vehicle 1 is, for example, an autonomous driving wheelchair that lets a user (i.e., passenger) 90 ride therein and travels autonomously. However, the autonomous driving vehicle 1 is not limited to a wheelchair but can also be a vehicle of a different type such as an automobile as long as the vehicle is a mobile object having the function of letting the user 90 ride therein and performing the autonomous driving. The distance (P3-P4) from the anxiety start position P3 to the passage start position P4 is a distance determined based on the speed of the autonomous driving vehicle 1 (i.e., vehicle speed), the anxiety factor 200, and an attribute of the user 90.

The autonomous driving vehicle 1 includes a vicinity sensor 20 that detects at least a situation in front in a traveling direction M, an information presentation unit 30 that presents information to the user 90, an operation unit (i.e., operation device or operation panel) 40 that outputs input information U based on a user operation, and a control device 10. Further, the autonomous driving vehicle 1 includes a drive unit 70 that drives wheels 71 and steering 72, a speed sensor 74, and a GPS (Global Positioning System) 51 as a positioning system. Furthermore, the autonomous driving vehicle 1 may include a storage device 50 and a condition grasping sensor 60 that detects condition of a person (i.e., the user, a subject, or an experiment participant) riding in the autonomous driving vehicle 1. The autonomous driving vehicle 1 may include a wireless communication device capable of connecting to an external network.

The vicinity sensor 20 includes, for example, a forward camera 21 as an image capturing device and a laser radar 22. It is permissible even if the vicinity sensor 20 includes only one of the forward camera 21 and the laser radar 22. The laser radar 22 is referred to also as a LiDAR (Light Detection and Ranging). The vicinity sensor 20 can also be a sensor of a different type as long as the sensor is capable of detecting an object situated in the vicinity including a region in front of the autonomous driving vehicle 1.

The information presentation unit 30 includes, for example, a display device 31 such as a liquid crystal display, a speaker 32 as an audio output device, a vibration device 33 that vibrates a seat 73 or the like on which the user 90 is seated, and an indicator lamp 34 such as an LED (Light-Emitting Diode) lamp. The information presentation unit 30 does not need to include all of the display device 31, the speaker 32, the vibration device 33 and the indicator lamp 34; it is permissible if the information presentation unit 30 includes one or more of these components.

The operation unit 40 can include a touch panel, a keyboard, an operation button, a control lever, and an audio input device capable of audio input, for example; however, it is permissible if the operation unit 40 includes one or more of these components. In cases where the operation unit 40 is a touch panel that receives a touch operation performed with a finger, the display device 31 and the operation unit 40 are formed integrally. The information presentation unit 30 and the operation unit 40 are referred to also as an HMI (Human Machine Interface).

The drive unit 70 includes, for example, a motor that rotates the wheels 71, a motor that drives the steering 72, and a drive circuit that controls the revolution of these motors. The speed sensor 74 detects the traveling speed of the autonomous driving vehicle 1.

The GPS 51 is a system that measures the position of the autonomous driving vehicle 1. The system measuring the position of the autonomous driving vehicle 1 is not limited to a GPS but can also be a positioning system of a different type.

The storage device 50 stores a variety of information. The storage device 50 can also be an external device included in a server or the like in a network capable of communication via a communication device. The storage device 50 stores, for example, road information R such as maps and road attributes, vehicle information V such as traveling performance and vehicle width (i.e., size) of the autonomous driving vehicle 1, vicinity detection information D obtained by the vicinity sensor 20, information collected by a collection unit 11, and so forth.

The condition grasping sensor 60 includes, for example, one or more of a face capturing camera, a whole body capturing camera, a stabilometer, a seat sensor, a tactile sensor, a pressure sensor, a different physiological measurement sensor, and so forth. The face capturing camera is a sensor for detecting a line of sight, a facial expression, or both of them of the person riding in the autonomous driving vehicle 1. The whole body capturing camera is a sensor for detecting posture and action of the person riding in the autonomous driving vehicle 1. The stabilometer is a sensor that measures fluctuation of the barycenter of the person riding in the autonomous driving vehicle 1. The seat sensor is a sensor arranged on the seat of the autonomous driving vehicle 1 for detecting the posture of the person seated on the seat. The physiological measurement sensor is, for example, a sensor that detects biological information regarding the person riding in the autonomous driving vehicle 1, such as heartbeat, pulsation and cutaneous electrical potential. It is also possible for the user to self-report (e.g., manually input) the anxiety through the operation unit 40, in which case the operation unit 40 functions as the condition grasping sensor 60. However, the anxiety condition of the user may also be judged automatically based on a detection signal from the condition grasping sensor 60. The face capturing camera as the condition grasping sensor 60 is, for example, an image capturing device that photographs the face of the user 90. The face capturing camera as the condition grasping sensor 60 photographs at least eyes 91 of the user 90. A control unit 12 is capable of detecting a sight line (line of sight) 92 of the user 90. The sight line 92 is represented by a straight line extending from the eyes 91 of the user 90 to the anxiety factor 200 as a target object viewed by the user 90. As a method of detecting the sight line 92, a publicly known technology (e.g., a technology described in Patent Reference 2 shown below) can be used.

Patent Reference 2: WO 2021/064791 A1.

The control device 10 includes the collection unit 11 that collects information and the control unit 12 that controls the operation of the entire device based on the collected information. The control device 10 is a computer, for example.

The collection unit 11 previously collects the anxiety start position P3, as a position before the anxiety factor 200 that makes an experiment participant feel anxiety in a route of autonomous traveling of the autonomous driving vehicle 1 with the experiment participant as a substitute for the user riding therein and a position where the experiment participant starts feeling anxiety about the anxiety factor 200, based on at least one of the experiment participant's operation on the operation unit 40 and a condition detection signal outputted from the condition grasping sensor 60 along the route (or depending on the route or in regard to each route).

Further, in regard to the anxiety start position P3, the collection unit 11 judges whether or not it is a situation (anxiety scene) where there exists an anxiety factor 200 on the travel route of the autonomous driving vehicle 1 and the passenger feels anxious “Is the autonomous driving wheelchair capable of passing through?”. In other words, at the anxiety start position P3, it is not a situation where the passenger of the autonomous driving vehicle 1 can confidently judge that the autonomous driving vehicle 1 is absolutely incapable/capable of passing through the position of the anxiety factor but a situation where the passenger of the autonomous driving vehicle 1 is thinking that the autonomous driving vehicle 1 can be either capable or incapable of passing through the position of the anxiety factor. That is, the collection unit 11 has a function as an anxiety determination unit that determines a position, where the passenger of the autonomous driving vehicle 1 starts having a feeling of anxiety that it is unclear whether the autonomous driving vehicle 1 is capable of passing through the position of the anxiety factor or not, based on the operations by the experiment participants.

Furthermore, the collection unit 11 may previously collect the recognition start position P1, as a position before the anxiety factor 200 that makes the experiment participant feel anxiety in the route of the autonomous traveling of the autonomous driving vehicle 1 with the experiment participant riding therein and a position where the experiment participant starts visually recognizing the existence of the anxiety factor 200, based on at least one of the experiment participant's operation on the operation unit 40 and the condition detection signal outputted from the condition grasping sensor 60, for example, in regard to each route. While the recognition start position P1 and the anxiety start position P3 are generally different positions, there are also cases where the recognition start position P1 equals the anxiety start position P3 (i.e., cases where the experiment participant has the feeling of anxiety immediately after visually recognizing the anxiety factor 200). The experiment participant is different from the user. It is desirable if there are a plurality of experiment participants. The experiment participants are desired to have a certain attribute coinciding with that of the user using the autonomous driving vehicle 1, such as sex the same as that of the user, a generation the same as that of the user, or appearance or preference similar to that of the user. It is permissible even if the experiment participant is the user.

The control unit 12 controls the operation of the autonomous driving vehicle 1 based on the road information R including a map of a scheduled route to the destination of the autonomous driving vehicle 1 and a road attribute of a road 80 of the scheduled route, the vehicle information V including the traveling performance and the vehicle width W1 of the autonomous driving vehicle 1, the vicinity detection information D outputted from the vicinity sensor 20, and the anxiety start position P3.

Further, the control unit 12 sets the presentation start position P2, as a position where the information presentation unit 30 is made to start presenting the anxiety reduction information, before the anxiety start position P3 in the route of the autonomous traveling while carrying the user 90. The distance (P2-P3) from the presentation start position P2 to the anxiety start position P3 is determined based on a time, as the sum total of a recognition time as a time necessary for the user 90 viewing the anxiety reduction information to recognize the contents of the anxiety reduction information and a buffer time for letting the recognition time have a margin, and the speed of the autonomous driving vehicle 1. Since each of the recognition start position P1, the presentation start position P2 and the anxiety start position P3 corresponds to a time as above, when the speed of the autonomous driving vehicle 1 is constant, the recognition start position P1, the presentation start position P2 and the anxiety start position P3 can be respectively regarded as a recognition start time point, a presentation start time point and an anxiety start time point. Further, in general, the control unit 12 sets the presentation start position P2 between the recognition start position P1 as the position where the experiment participant starts visually recognizing the existence of the anxiety factor 200 and the anxiety start position P3 or at the same position as the recognition start position P1. The control unit 12 makes the information presentation unit 30 start the presentation of the anxiety reduction information when the autonomous driving vehicle 1 with the user 90 riding therein reaches the presentation start position P2. It is permissible even if the presentation start position P2 is previously calculated based on the anxiety start position P3 and stored in a storage device usable by the autonomous driving vehicle 1.

Furthermore, the control unit 12 may judge that there exists an anxiety factor when the user 90 keeps on gazing at something for a time longer than or equal to a predetermined reference time based on face images captured by the face capturing camera as the condition grasping sensor 60.

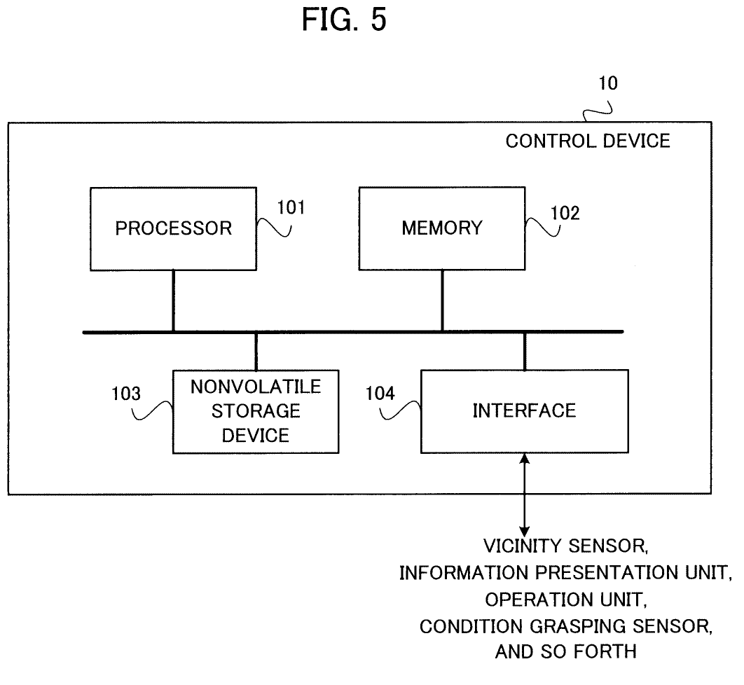

FIG. 5 is a diagram showing an example of the hardware configuration of the control device 10 of the autonomous driving vehicle 1 according to the first embodiment. The control device 10 is a device capable of executing a control method according to the first embodiment. As shown in FIG. 5, the control device 10 includes a processor 101 such as a CPU (Central Processing Unit), a memory 102 as a volatile storage device, a nonvolatile storage device 103 such as a hard disk drive (HDD) or a solid state drive (SSD), and an interface 104. The memory 102 is, for example, a semiconductor memory such as a RAM (Random Access Memory). The nonvolatile storage device 103 can be a device the same as the storage device 50 shown in FIG. 1.

Functions of the control device 10 are implemented by processing circuitry, for example. The processing circuitry can be either dedicated hardware or the processor 101 executing a program stored in the memory 102 as a storage device (i.e., record medium). The storage device may be a non-transitory computer-readable storage medium, namely, a non-transitory tangible storage medium storing a program such as the recommended activity presentation program. The processor 101 can be any one of a processing device, an arithmetic device, a microprocessor, a microcomputer and a DSP (Digital Signal Processor).

In the case where the processing circuitry is dedicated hardware, the processing circuitry is, for example, a single circuit, a combined circuit, a programmed processor, a parallelly programmed processor, an ASIC (Application Specific Integrated Circuit), an FPGA (Field-Programmable Gate Array) or a combination of some of these circuits.

In the case where the processing circuitry is the processor 101, a control program of the autonomous driving vehicle 1 is implemented by software, firmware, or a combination of software and firmware. The control program is installed in the control device 10 via a network or from a record medium. The software and the firmware are described as programs and stored in the memory 102. The processor 101 implements the functions of the units shown in FIG. 1 by reading out and executing the control program stored in the memory 102.

Incidentally, it is also possible to implement part of the control device 10 by dedicated hardware and the other part by software or firmware. As above, the processing circuitry is capable of implementing the above-described functions by hardware, software, firmware, or a combination of some of these means.

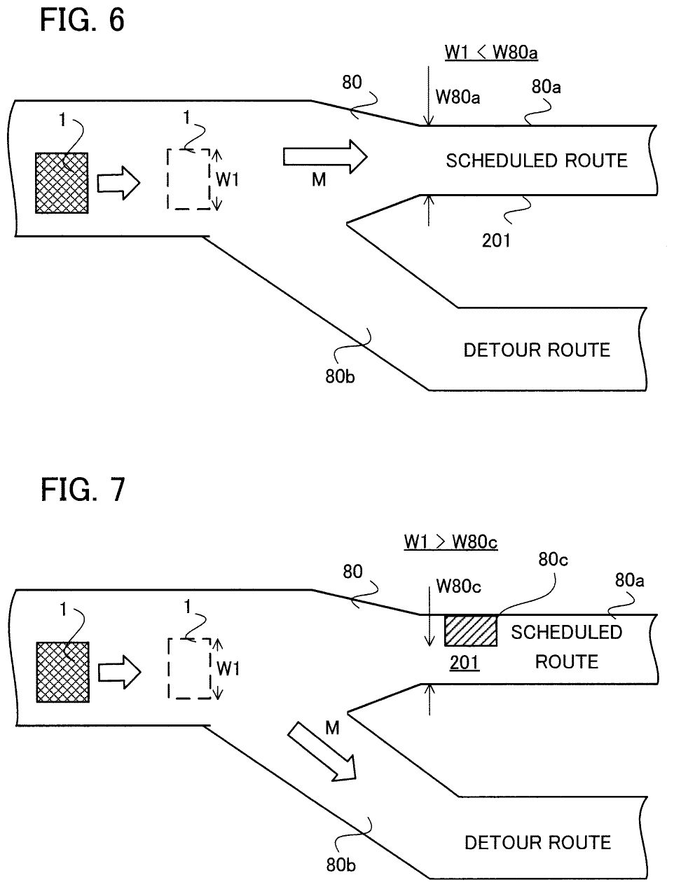

FIG. 6 is a schematic plan view showing an example in which the autonomous driving vehicle 1 travels on a scheduled route 80a even though there exists a narrow road width as an anxiety factor 201. FIG. 7 is a schematic plan view showing an example in which there exists a narrow road width as the anxiety factor 201 and the autonomous driving vehicle 1 travels on a detour route 80b. The control unit 12 acquires a passable road width (W80a in FIG. 6, W80c in FIG. 7) in the scheduled route 80a as the anxiety factor 201 from the road information R, the vicinity detection information D, or both of the road information R and the vicinity detection information D, makes a judgment based on the road width and the vehicle width W1, and operates based on the result of the judgment.

Specifically, for example, when the road width W80a in the scheduled route 80a is wider than the vehicle width W1 by a predetermined margin value Wa or more, the control unit 12 judges that the scheduled route 80a is passable. That is, as shown in FIG. 6, the control unit 12 judges that the scheduled route 80a is passable if W1<W80a and (W80a−W1)≥Wa. The margin value Wa is not limited to a fixed value but can also be a value that varies depending on the vehicle speed and the road attribute (e.g., a road inclination, road surface condition, or both of these). For example, the margin value Wa can vary so as to increase with the increase in the vehicle speed. Further, the margin value Wa can vary so as to increase with the increase in the road inclination (inclination in the transverse direction). Furthermore, the margin value Wa can vary so as to increase with the increase in deterioration of the road surface condition of the road. Moreover, the margin value Wa can vary so as to increase with the increase in the size of a step (level difference) on the road. In addition, the margin value Wa can vary so as to increase with the increase in a road gradient (inclination in the traveling direction).

Further, the control unit 12 judges that the scheduled route 80a is impassable when an object (e.g., construction area) 80c exists on the scheduled route 80a and the road width W80c of the usable road is less than or equal to the vehicle width W1, for example. That is, as shown in FIG. 7, if W1≥W80c, the control unit 12 judges that the scheduled route 80a is impassable and changes the scheduled route to the detour route 80b. In other words, the control unit 12 judges whether the autonomous driving vehicle 1 is capable of passing through the position of the anxiety factor 201 or not based on the road width W80 and the vehicle width W1, and when the autonomous driving vehicle 1 is judged to be incapable of passing through the position of the anxiety factor 201, changes the scheduled route so as to detour around the position of the anxiety factor 201.

In each of the case where the scheduled route is changed and the case where the scheduled route is not changed, the control unit 12 presents the anxiety reduction information at the previously set presentation start position P2.

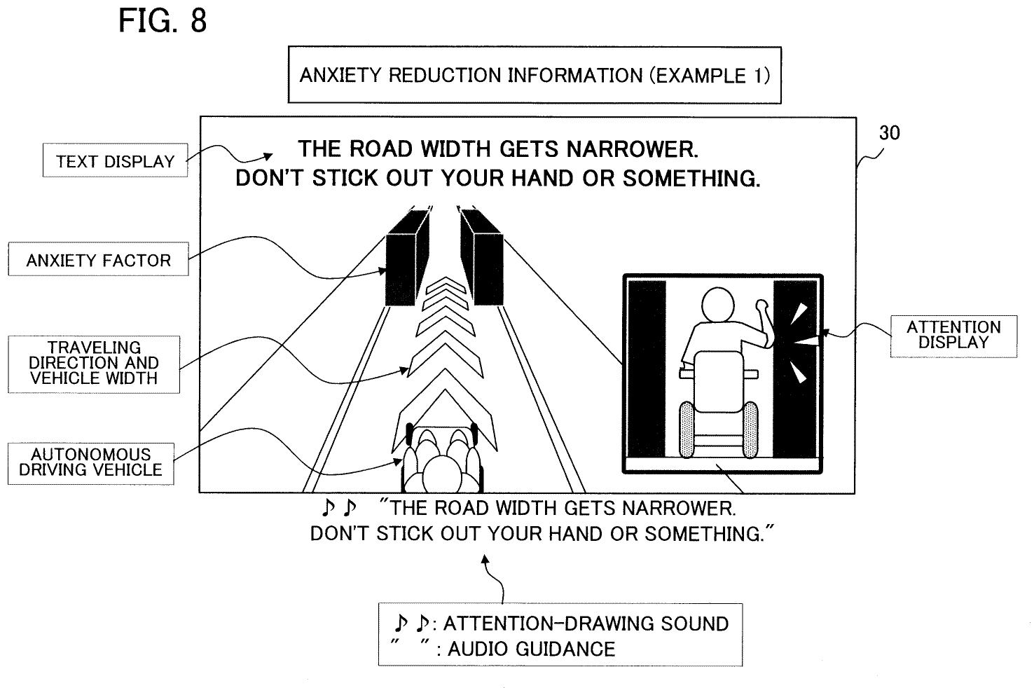

FIG. 8 is a diagram showing an example 1 of the anxiety reduction information presented by the information presentation unit 30 of the autonomous driving vehicle 1. FIG. 8 shows an example of the anxiety reduction information (an example of a display image, an attention-drawing sound and audio guidance) in a case where the anxiety factor 201 is a narrow road width whereas the road width is wider than the vehicle width W1 of the autonomous driving vehicle 1 and the autonomous driving vehicle 1 travels on the scheduled route. In this example, a display and audio guidance “THE ROAD GETS NARROWER. DON'T STICK OUT YOUR HAND OR SOMETHING.” and an attention display (a figure indicating a prohibited action of the user) are shown.

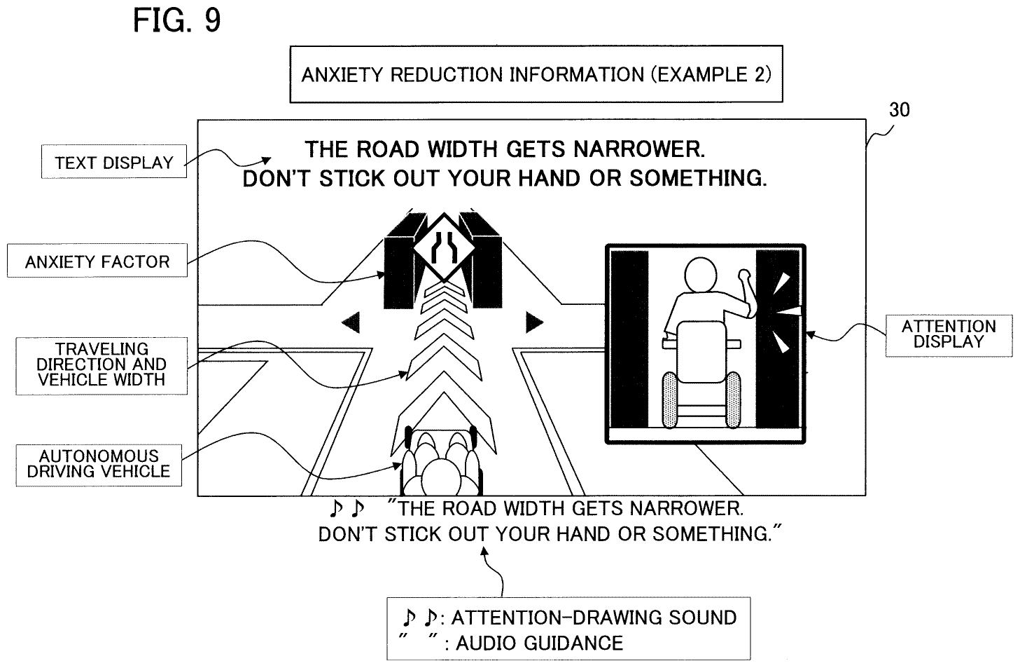

FIG. 9 is a diagram showing an example 2 of the anxiety reduction information presented by the information presentation unit 30 of the autonomous driving vehicle 1. FIG. 9 shows an example of the anxiety reduction information (an example of the display image, the attention- drawing sound and the audio guidance) in a case where the anxiety factor 201 is a narrow road width whereas the road width is wider than the vehicle width W1 of the autonomous driving vehicle 1 and the autonomous driving vehicle 1 travels on the scheduled route. In this example, the display and the audio guidance “THE ROAD GETS NARROWER. DON'T STICK OUT YOUR HAND OR SOMETHING.”, the attention display (the figure indicating the prohibited action of the user), and a figure indicating how the road width gets narrower in superimposition with the anxiety factor 201 are shown.

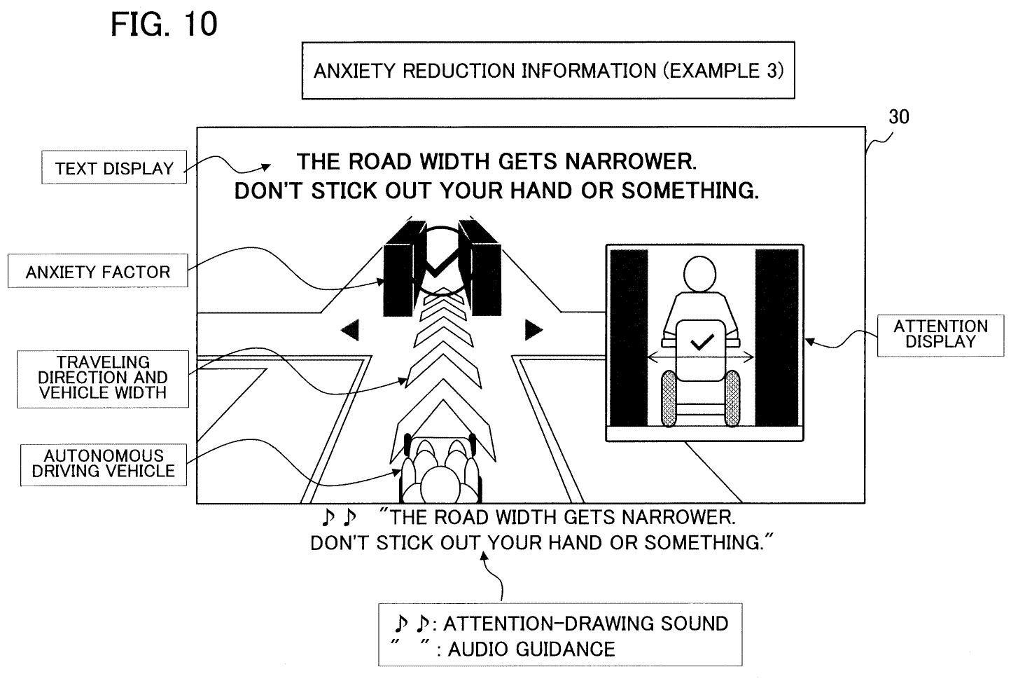

FIG. 10 is a diagram showing an example 3 of the anxiety reduction information presented by the information presentation unit 30 of the autonomous driving vehicle 1. FIG. 10 shows an example of the anxiety reduction information (an example of the display image, the attention-drawing sound and the audio guidance) in a case where the anxiety factor 201 is a narrow road width whereas the road width is wider than the vehicle width W1 of the autonomous driving vehicle 1 and the autonomous driving vehicle 1 travels on the scheduled route. In this example, the display and the audio guidance “THE ROAD GETS NARROWER. DON'T STICK OUT YOUR HAND OR SOMETHING.”, an attention display (a figure indicating a desirable posture that the user should assume), and a figure as a mark (check mark) indicating the position where the road width gets narrower displayed in superimposition with the anxiety factor 201 are shown.

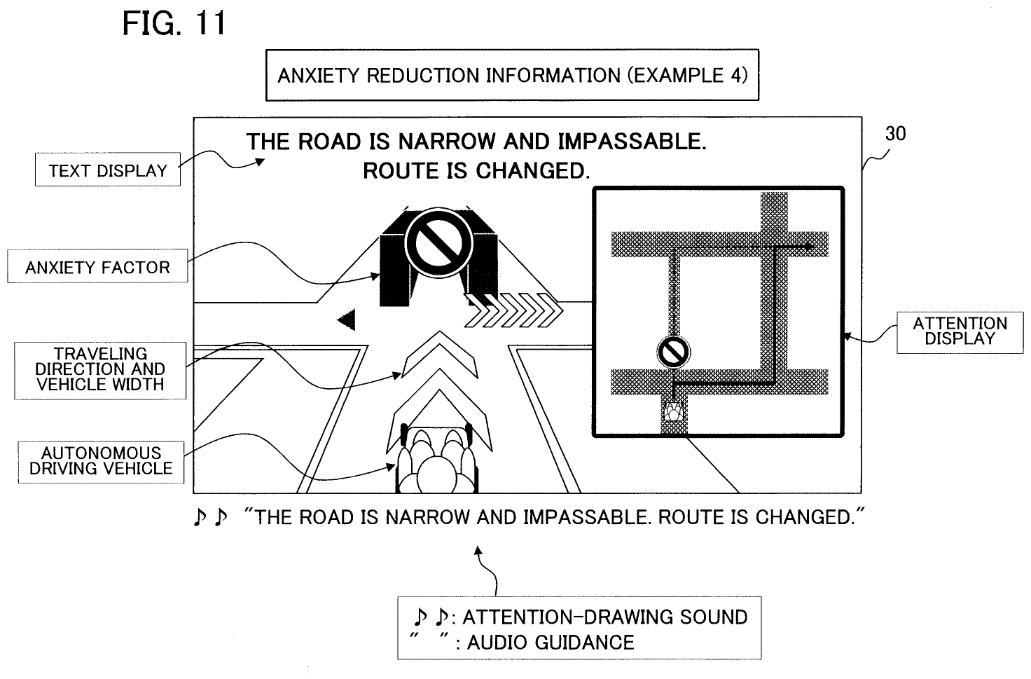

FIG. 11 is a diagram showing an example 4 of the anxiety reduction information presented by the information presentation unit 30 of the autonomous driving vehicle 1. FIG. 11 shows an example of the anxiety reduction information (an example of the display image, the attention-drawing sound and the audio guidance) in a case where the anxiety factor 201 is a narrow road width and the autonomous driving vehicle 1 travels on a detour route (in FIG. 11, a route making a right turn) without traveling on the scheduled route. In this example, a display and audio guidance “THE ROAD IS NARROW AND IMPASSABLE. ROUTE IS CHANGED.”, an attention display (a figure indicating a desirable posture that the user should assume), the figure as the no-entry mark indicating that it is impossible to enter the position of the anxiety factor 201 displayed in superimposition with the anxiety factor 201, and a figure indicating that the detour route is a route making a right turn are shown.

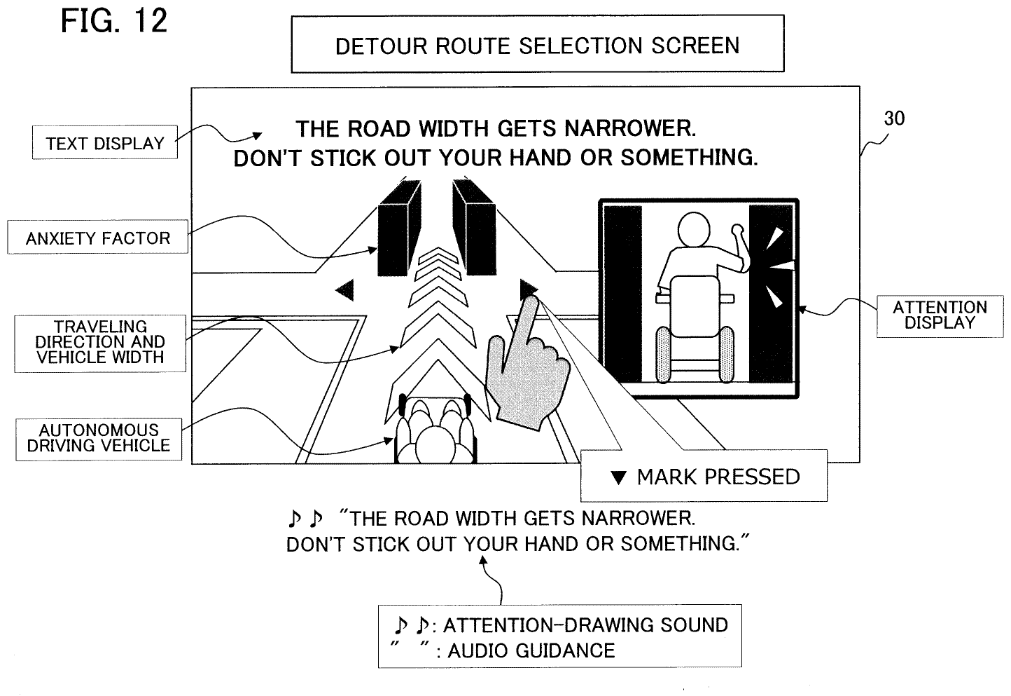

FIG. 12 is a diagram showing a detour route selection screen presented by the information presentation unit 30 of the autonomous driving vehicle 1. FIG. 12 shows an example of the anxiety reduction information (an example of the display image, the attention-drawing sound and the audio guidance) in a case where the anxiety factor 201 is a narrow road width and the autonomous driving vehicle 1 is capable of traveling on the scheduled route whereas the autonomous driving vehicle 1 travels on a detour route (in FIG. 12, the route making a right turn) according to an operation by the user (in this example, an operation of touching a triangular mark on the touch panel). In this example, the display and the audio guidance “THE ROAD GETS NARROWER. DON'T STICK OUT YOUR HAND OR SOMETHING.”, the attention display (the figure indicating the prohibited action of the user), the figure as the no-entry mark indicating that it is impossible to enter the position of the anxiety factor 201 displayed in superimposition with the anxiety factor 201, and the figure indicating that the detour route is a route making a right turn are shown.

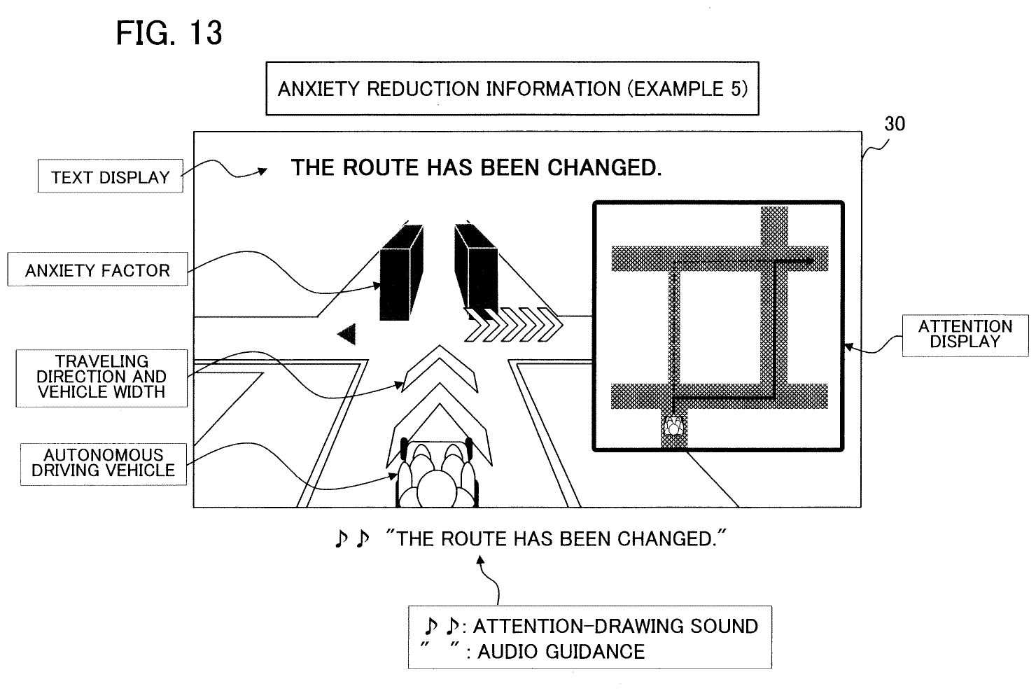

FIG. 13 is a diagram showing an example 5 of the anxiety reduction information presented by the information presentation unit 30 after the detour route (right turn) is selected on the screen of FIG. 12. FIG. 13 shows an example of the anxiety reduction information (an example of the display image, the attention-drawing sound and the audio guidance) in a case where the autonomous driving vehicle 1 travels on the detour route after the detour route (right turn) is selected. In this example, a display and audio guidance “THE ROUTE HAS BEEN CHANGED.”, an attention display (a figure indicating the detour route), and the figure indicating that the detour route is a route making a right turn are shown. As above, when there are a plurality of detour routes (e.g., a right turn and a left turn), one of the plurality of detour routes can be selected by the user 90's operation on the operation unit 40 (touch panel in the example of FIG. 12 and FIG. 13).

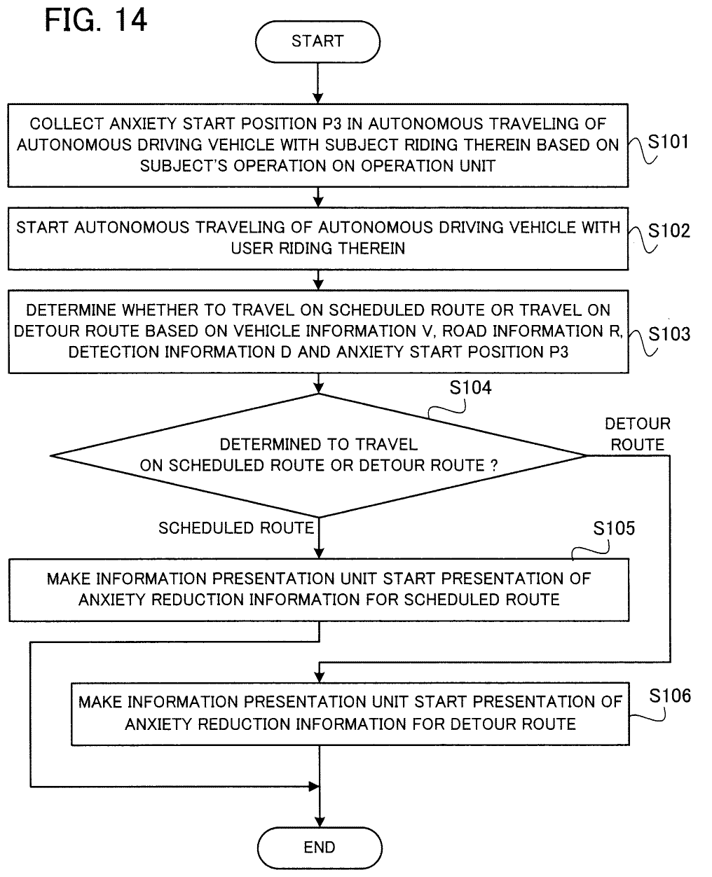

FIG. 14 is a flowchart showing the operation of the control device 10 of the autonomous driving vehicle 1 according to the first embodiment. First, the collection unit 11 of the control device 10 collects the anxiety start position P3 in the autonomous traveling of the autonomous driving vehicle 1 with an experiment participant riding therein based on operation on the operation unit 40 by the experiment participant and stores the collected anxiety start position P3 in the storage device 50 (step S101 in FIG. 14). When there are a plurality of collection results, the collection unit 11 determines a position represented by using a representative value, such as the mean value, the median, the maximum value or the minimum value, of the distance (or the time) from the anxiety factor as the anxiety start position P3, for example.

Subsequently, the control unit 12 of the control device 10 starts the autonomous traveling of the autonomous driving vehicle 1 in which the user is riding (step S102 in FIG. 14). Here, the autonomous driving vehicle 1 in which the user is riding is desired to be the same as the autonomous driving vehicle in which the experiment participant rode in the shape and functions (be of the same model) but does not need to be physically identical with the autonomous driving vehicle in which the experiment participant rode. It is permissible if the autonomous driving vehicle in which the experiment participant rides is of a model the same as or similar to the model of the autonomous driving vehicle 1 in which the user 90 rides and is capable of using information regarding the anxiety start position P3 collected by the collection unit 11 and determined in the step S101.

Subsequently, the control unit 12 determines whether to travel on the scheduled route 80a or travel into the detour route 80b, namely, whether or not to travel on the scheduled route 80a, based on the vehicle width W1 (the vehicle information V) and the road width W80a, W80c (the road information R or the vicinity detection information D) (step S103 in FIG. 14).

When the autonomous driving vehicle 1 travels on the scheduled route 80a as shown in FIG. 6 (step S104 in FIG. 14), the control unit 12 starts the presentation of the anxiety reduction information shown in any one of FIG. 8 to FIG. 10 at the presentation start position P2 determined based on the anxiety start position P3 (step S105).

When the autonomous driving vehicle 1 travels into the detour route 80b as shown in FIG. 7 (step S104 in FIG. 14), the control unit 12 starts the presentation of the anxiety reduction information shown in FIG. 11 at the presentation start position P2 determined based on the anxiety start position P3 (step S106).

As described above, according to the first embodiment, the presentation of the anxiety reduction information is started at the presentation start position P2 determined based on the anxiety start position P3, and thus the presentation of the anxiety reduction information can be started even in a period in which the movement of the autonomous driving vehicle 1 does not change and the physiological index is hardly changed. Therefore, the anxiety reduction effect on the user 90 can be enhanced. Further, since the presentation start position P2 as the start position of the presentation of the anxiety reduction information is determined based on information previously collected based on reports from the experiment participants, presentation of unnecessary information at positions where the presentation is unnecessary can be reduced.

Specifically, there are cases where there occurs a situation where the user 90 has a feeling of anxiety “Is the autonomous traveling of the autonomous driving vehicle I'm riding in capable of traveling of the autonomous driving vehicle through a route in front in the traveling direction?”. The autonomous driving vehicle 1 in the first embodiment is capable of notifying the user 90 of the information for reducing the user 90's feeling of anxiety at the presentation start position P2 before the anxiety start position P3 as the position where there occurs a situation in which the user 90 has the feeling of anxiety.

### Second Embodiment

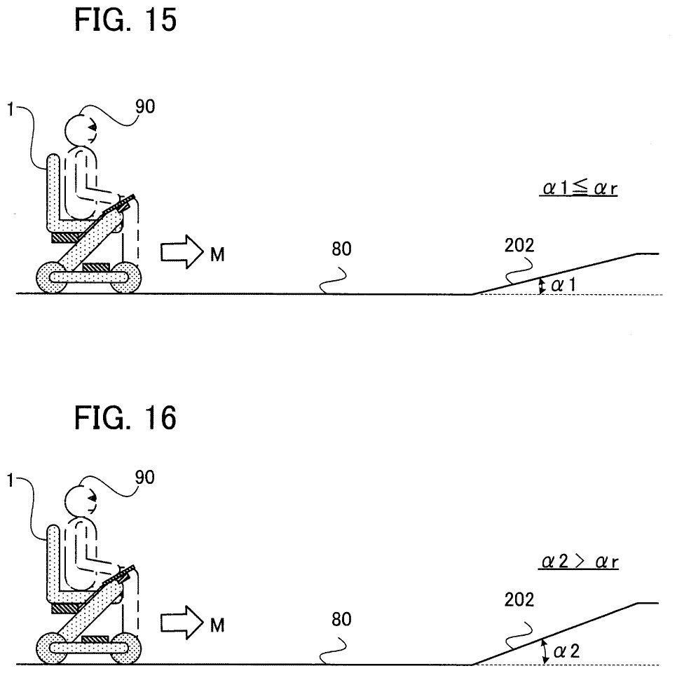

In a second embodiment, a description will be given of an example in which the anxiety factor is a slope as a road surface condition in the scheduled route. FIG. 1 and FIG. 5 are also referred to in the description of the second embodiment. FIG. 15 is a schematic diagram showing an example in which the autonomous driving vehicle 1 according to the second embodiment travels on the scheduled route even though there exists a slope as an anxiety factor 202. FIG. 16 is a schematic diagram showing an example in which there exists a slope as the anxiety factor 202 and the autonomous driving vehicle 1 according to the second embodiment avoids the scheduled route (i.e., travels into a different route).

In the second embodiment, the control unit 12 judges whether the autonomous driving vehicle 1 is capable of passing through the slope as the anxiety factor 202 or not based on the road surface condition and the traveling performance (climbing performance in this example), and when the autonomous driving vehicle 1 is judged to be incapable of passing through the slope, changes the scheduled route so as to detour around the position of the anxiety factor 202 and executes a process for making the information presentation unit 30 present the user with the fact that the slope is impassable at the presentation start position P2 (FIG. 4). The case where the slope is judged to be impassable can be, for example, a case where an inclination angle α2 of the slope is greater than a passable inclination angle αr represented by the traveling performance as shown in FIG. 16. In this case, the control unit 12 makes the information presentation unit 30 present information indicating that the scheduled route is changed. While an angle (inclination angle) has been taken as an example, it is also possible to set a plurality of factors influencing the climbing performance, such as the user's body weight and the traveling speed, as a plurality of parameters influencing the climbing performance. Except for these features, the configuration in the second embodiment is the same as the configuration in the first embodiment.

Further, in the second embodiment, when the slope is judged to be passable based on the road surface condition and the traveling performance, the control unit 12 does not change the scheduled route. The case where the slope is judged to be passable can be, for example, a case where an inclination angle α1 of the slope is less than or equal to the passable inclination angle αr represented by the traveling performance as shown in FIG. 15. In this case, the control unit 12 acquires the inclination angle α1 of the slope as the anxiety factor 202 from the road information R or the vicinity detection information D and makes the information presentation unit 30 present that the slope is passable at the presentation start position P2 (FIG. 4) based on the road surface condition (the inclination of the slope in this example) and the traveling performance of the autonomous driving vehicle 1. While examples in which the slope is an upward slope are shown in FIG. 15 and FIG. 16, a similar judgment is made also in cases where the slope is a downward slope.

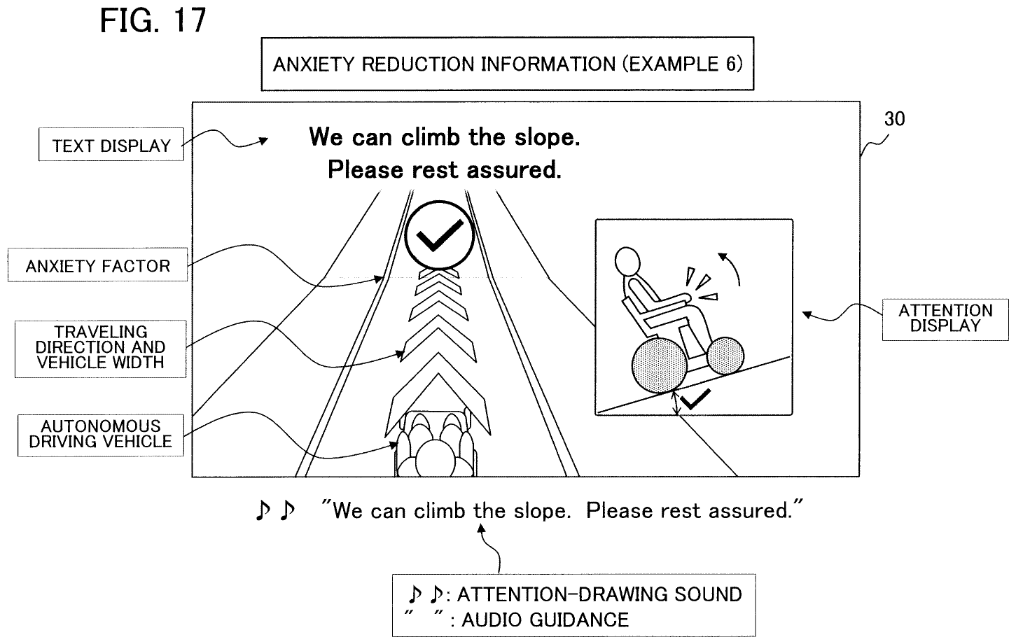

FIG. 17 is a diagram showing an example 6 of the anxiety reduction information presented by the information presentation unit 30 of the autonomous driving vehicle 1. FIG. 17 shows an example of the anxiety reduction information (an example of the display image, the attention-drawing sound and the audio guidance) in a case where the anxiety factor 202 is an upward slope (i.e., a sloping surface having a rising gradient) whereas the inclination angle α1 of the sloping surface is less than or equal to the inclination angle αr passable with the traveling performance of the autonomous driving vehicle 1 and the autonomous driving vehicle 1 travels on the scheduled route. In this example, an example of presenting “We can climb the slope. Please rest assured.” in English is shown.

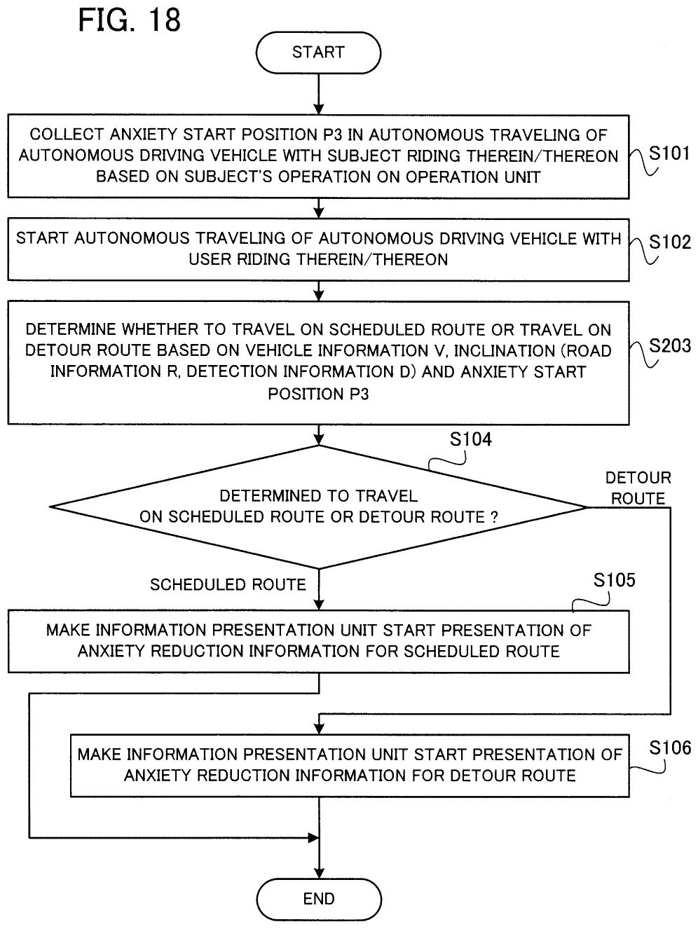

FIG. 18 is a flowchart showing the operation of the control device 10 of the autonomous driving vehicle 1 according to the second embodiment. In FIG. 18, each step being the same as a step shown in FIG. 14 is assigned the same reference character as in FIG. 14. The autonomous driving vehicle 1 according to the second embodiment differs from the autonomous driving vehicle 1 in the first embodiment (that makes the determination based on the road width and the vehicle width) in that the control unit 12 in step S203 determines whether to travel on the scheduled route or travel into the detour route based on the traveling performance (the vehicle information V) and the inclination angle of the slope (the road information R or the vicinity detection information D). Except for this feature, the operation in FIG. 18 is the same as the operation in FIG. 14.

As described above, according to the second embodiment, the presentation of the anxiety reduction information is started at the presentation start position P2 determined based on the anxiety start position P3, and thus the presentation of the anxiety reduction information can be started even in a period in which the movement of the autonomous driving vehicle 1 does not change and the physiological index is hardly changed. Therefore, the anxiety reduction effect on the user 90 can be enhanced. Further, since the presentation start position P2 as the start position of the presentation of the anxiety reduction information is determined based on information previously collected based on reports from the experiment participants, the presentation of unnecessary information at positions where the presentation is unnecessary can be reduced.

Furthermore, it is also possible to incorporate the configuration and functions of the autonomous driving vehicle 1 according to the second embodiment into the autonomous driving vehicle according to the first embodiment.

### Third Embodiment

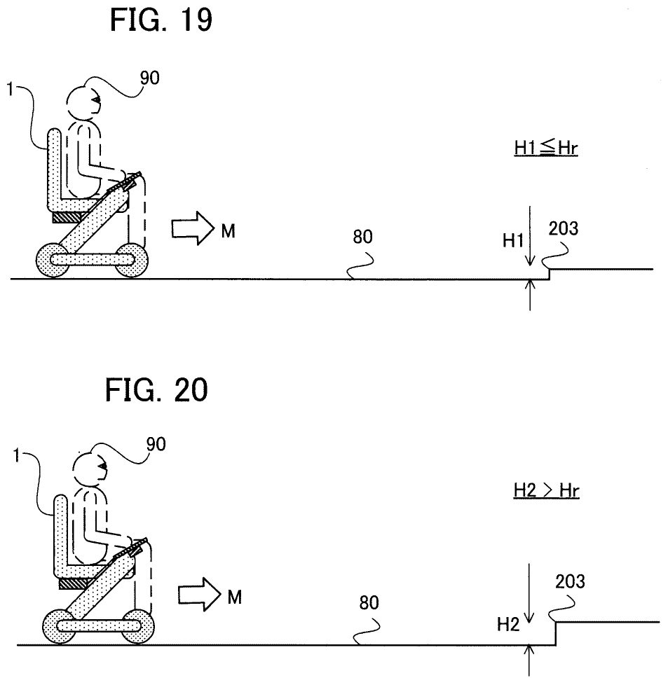

In a third embodiment, a description will be given of an example in which the anxiety factor is a step as a road surface condition in the scheduled route. FIG. 1 and FIG. 5 are also referred to in the description of the third embodiment. FIG. 19 is a schematic diagram showing an example in which the autonomous driving vehicle 1 according to the third embodiment travels on the scheduled route even though there exists a step as an anxiety factor 203. FIG. 20 is a schematic diagram showing an example in which there exists a step as the anxiety factor 203 and the autonomous driving vehicle 1 according to the third embodiment avoids the scheduled route (i.e., travels into a different route).

In the third embodiment, the control unit 12 judges whether the autonomous driving vehicle 1 is capable of passing through the step as the anxiety factor 203 or not based on the road surface condition and the traveling performance, and when the autonomous driving vehicle 1 is judged to be incapable of passing through the step, changes the scheduled route so as to detour around the position of the anxiety factor 203 and executes a process for making the information presentation unit 30 present the user with the fact that the step is impassable at the presentation start position P2 (FIG. 4). The case where the step is judged to be impassable can be, for example, a case where a height H2 of the step is greater than a passable step height Hr represented by the traveling performance as shown in FIG. 20. In this case, the control unit 12 makes the information presentation unit 30 present information indicating that the scheduled route is changed. Except for these features, the configuration in the third embodiment is the same as the configuration in the first embodiment.

Further, in the third embodiment, when the step is judged to be passable based on the road surface condition and the traveling performance, the control unit 12 does not change the scheduled route. The case where the step is judged to be passable can be, for example, a case where a height H1 of the step is less than or equal to the passable step height Hr represented by the traveling performance as shown in FIG. 19. In this case, the control unit 12 acquires the height H1 of the step as the anxiety factor 203 from the road information R, the vicinity detection information D, or both of the road information R and the vicinity detection information D, and makes the information presentation unit 30 present that the step is passable at the presentation start position P2 (FIG. 4) based on the road surface condition (the height of the step in this example) and the traveling performance of the autonomous driving vehicle 1. While examples in which the step is an upward step are shown in FIG. 19 and FIG. 20, a similar judgment is made also in cases where the step is a downward step.

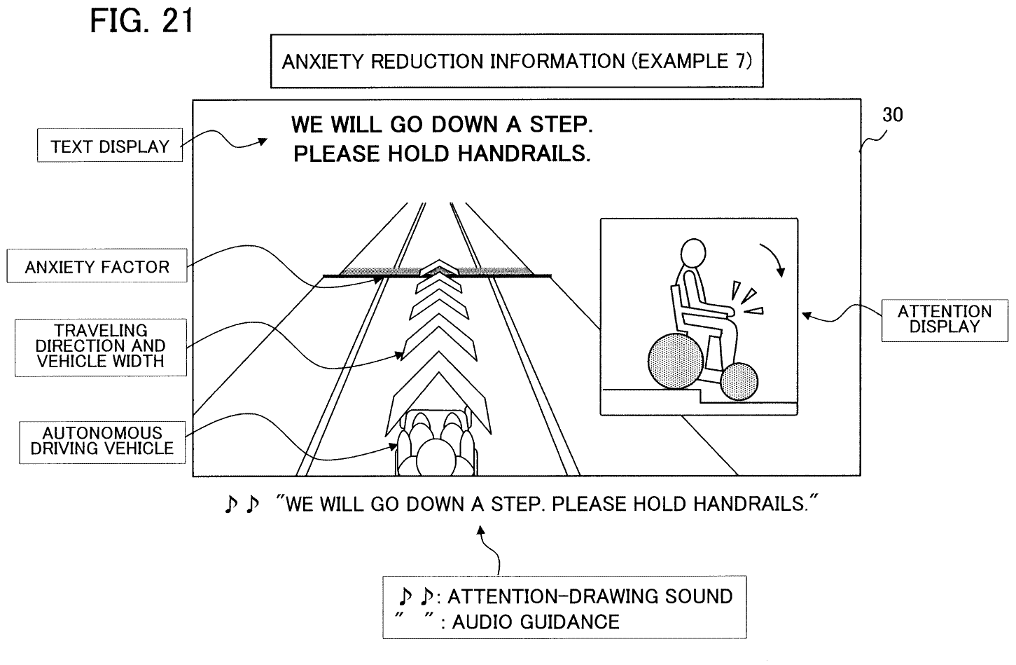

FIG. 21 is a diagram showing an example 7 of the anxiety reduction information presented by the information presentation unit 30 of the autonomous driving vehicle 1. FIG. 21 shows an example of the anxiety reduction information (an example of the display image, the attention-drawing sound and the audio guidance) in a case where the anxiety factor 203 is a downward step whereas the height of the step is less than or equal to the height passable with the traveling performance of the autonomous driving vehicle 1 and the autonomous driving vehicle 1 travels on the scheduled route. In this example, a display and audio guidance “WE WILL GO DOWN A STEP. PLEASE HOLD HANDRAIL.” and an attention display (a figure prompting the user to pay attention) are shown.

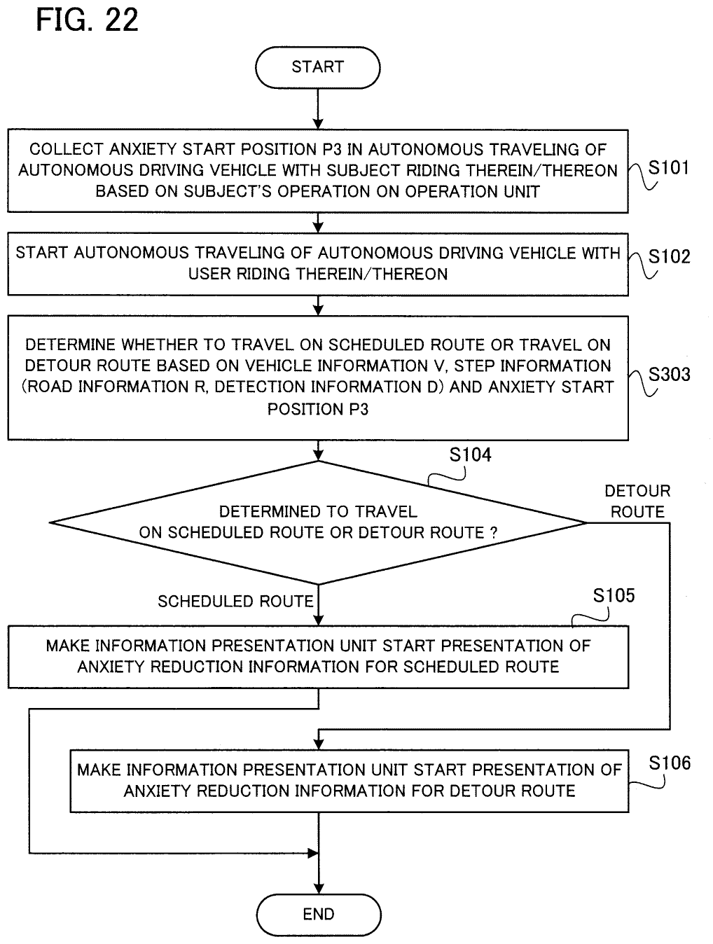

FIG. 22 is a flowchart showing the operation of the control device 10 of the autonomous driving vehicle 1 according to the third embodiment. In FIG. 22, each step being the same as a step shown in FIG. 14 is assigned the same reference character as in FIG. 14. The autonomous driving vehicle 1 according to the third embodiment differs from the autonomous driving vehicle 1 in the first embodiment in that the control unit 12 in step S303 determines whether to travel on the scheduled route or travel into the detour route based on the traveling performance (the vehicle information V) and the height of the step (the road information R or the vicinity detection information D). Except for this feature, the operation in FIG. 22 is the same as the operation in FIG. 14.

As described above, according to the third embodiment, the presentation of the anxiety reduction information is started at the presentation start position P2 determined based on the anxiety start position P3, and thus the presentation of the anxiety reduction information can be started even in a period in which the movement of the autonomous driving vehicle 1 does not change and the physiological index is hardly changed. Therefore, the anxiety reduction effect on the user 90 can be enhanced. Further, since the presentation start position P2 as the start position of the presentation of the anxiety reduction information is determined based on information previously collected based on reports from the experiment participants, the presentation of unnecessary information at positions where the presentation is unnecessary can be reduced.

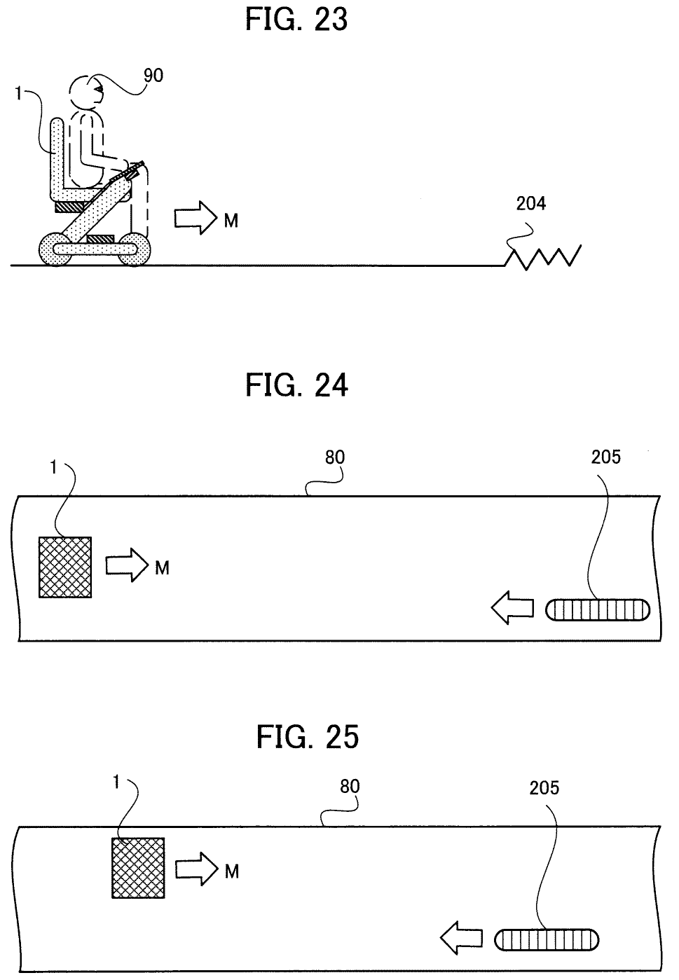

FIG. 23 shows a modification of the third embodiment. FIG. 23 is a schematic diagram showing an example in which the user riding in the autonomous driving vehicle 1 has recognized unevenness (i.e., an uneven surface not being flat) as an anxiety factor 204. In this case, similarly to the cases of FIG. 19 and FIG. 20, the control unit 12 judges whether the road on which the unevenness exists is passable or not based on height or depth of the unevenness (i.e., magnitude of the unevenness) and determines the presentation start position P2 as the start position of the presentation of the anxiety reduction information based on the information collected based on reports from the experiment participants, by which the presentation of unnecessary information at positions where the presentation is unnecessary can be reduced.

Further, when the anxiety factor is an unpaved surface (e.g., an area with grass, a marshy place, damp ground or the like) existing in the scheduled route, the control unit 12 judges whether the road on which the unpaved surface exists is passable or not based on condition of the unpaved surface (e.g., the amount of the grass, the width of the marshy place, or the like) and determines the presentation start position P2 as the start position of the presentation of the anxiety reduction information based on the information collected based on reports from the experiment participants, by which the presentation of unnecessary information at positions where the presentation is unnecessary can be reduced.

Furthermore, it is also possible to incorporate the configuration and functions of the autonomous driving vehicle 1 according to the third embodiment into the autonomous driving vehicle according to the first or second embodiment.

### Fourth Embodiment

In a fourth embodiment, a description will be given of an example in which the anxiety factor is a mobile object that approaches the autonomous driving vehicle in the scheduled route. FIG. 1 and FIG. 5 are also referred to in the description of the fourth embodiment. FIGS. 24 and 25 are schematic plan views showing the operation of the autonomous driving vehicle 1 according to the fourth embodiment when a mobile object as an anxiety factor 205 approaches the autonomous driving vehicle 1.

In the fourth embodiment, the control unit 12 regulates the autonomous traveling of the autonomous driving vehicle 1 based on the size and the speed of the mobile object (the vicinity detection information D) approaching the autonomous driving vehicle 1 and the distance to the mobile object. The mobile object is detected based on the vicinity detection information D from the vicinity sensor 20. The mobile object is, for example, a pedestrian, a bicycle, an automobile or the like. As shown in FIG. 25, the autonomous driving vehicle 1 shifts the traveling position of the autonomous driving vehicle 1 closer to the side strip of the road so as to separate from the lane in which the mobile object is traveling. In this case, the control unit 12 may make the information presentation unit 30 present the user 90 with the fact that the mobile object as the anxiety factor 205 has already been recognized and the scheduled travel route has been shifted to the position closer to the side strip.

For example, the control unit 12 judges that a gaze target at which the user 90 keeps on gazing for a time longer than or equal to a predetermined reference time based on face images captured by the face capturing camera as the condition grasping sensor 60 is the anxiety factor 205, and when the gaze target is judged to be a mobile object as a moving object based on the vicinity detection information D from the vicinity sensor 20, modifies the scheduled route so as to separate from the position of the anxiety factor 205 and executes a process for making the information presentation unit 30 present the user with the fact that the autonomous driving vehicle 1 passes by the mobile object soon at the presentation start position P2 (FIG. 4).

Further, the control unit 12 may control the information presentation unit 30 so as to increase an enhancement level of a display mode of the information regarding the mobile object with the increase in the speed of the mobile object. To increase the enhancement level of the display mode means, for example, for the display device 31 to show the display by using larger letters, show the display by using more conspicuous colors, blink the display, increase the luminance of the display, or the like. To increase the enhancement level of the display mode means, for example, for the speaker 32 to output louder sound, output iterative sound, or the like. To increase the enhancement level of the display mode means, for example, for the vibration device 33 to give stronger vibration, give iterative vibration, or the like. To increase the enhancement level of the display mode means, for example, for the indicator lamp 34 to show the display brighter, show the display by using a more conspicuous color, blink the display, or the like. To increase the enhancement level of the display mode can also be to combine together the display modes of two or more of the display device 31, the speaker 32, the vibration device 33 and the indicator lamp 34.

In the process of collecting information, the collection unit 11 may collect anxiety strength levels inputted by the experiment participant through the operation unit 40 between the recognition start position P1 to the anxiety factor in the autonomous traveling of the autonomous driving vehicle 1 with the experiment participant riding therein, and the control unit 12 may increase the enhancement level of the display mode of the information presented by the information presentation unit 30 with the increase in the anxiety strength level.

Further, the control unit 12 may increase the enhancement level of the display mode of the information regarding the mobile object with the increase in the size of the mobile object. Furthermore, the control unit 12 may increase the enhancement level of the display mode of the information regarding the approaching mobile object with the decrease in the distance to the mobile object. Except for the above-described features, the configuration in the fourth embodiment is the same as the configuration in the first embodiment.

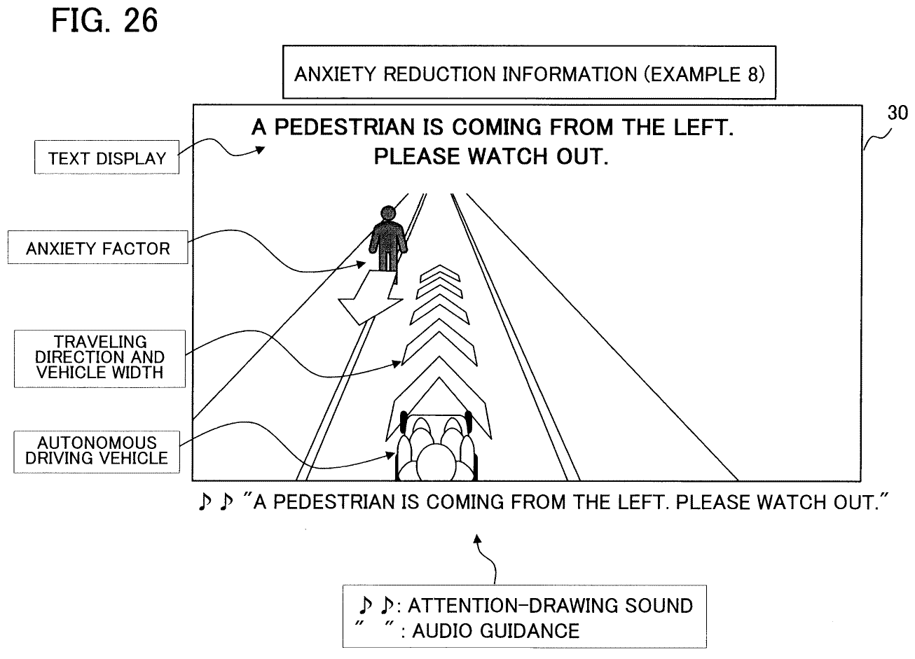

FIG. 26 is a diagram showing an example 8 of the anxiety reduction information presented by the information presentation unit 30 of the autonomous driving vehicle 1. FIG. 26 shows an example of the anxiety reduction information (an example of the display image, the attention-drawing sound and the audio guidance) in a case where the anxiety factor 204 is a pedestrian as a mobile object whereas the pedestrian is walking on the roadside not affecting the traveling of the autonomous driving vehicle 1 and the autonomous driving vehicle 1 travels on the scheduled route. In this example, a display and audio guidance “A PEDESTRIAN IS COMING FROM THE LEFT. PLEASE WATCH OUT.” are shown.

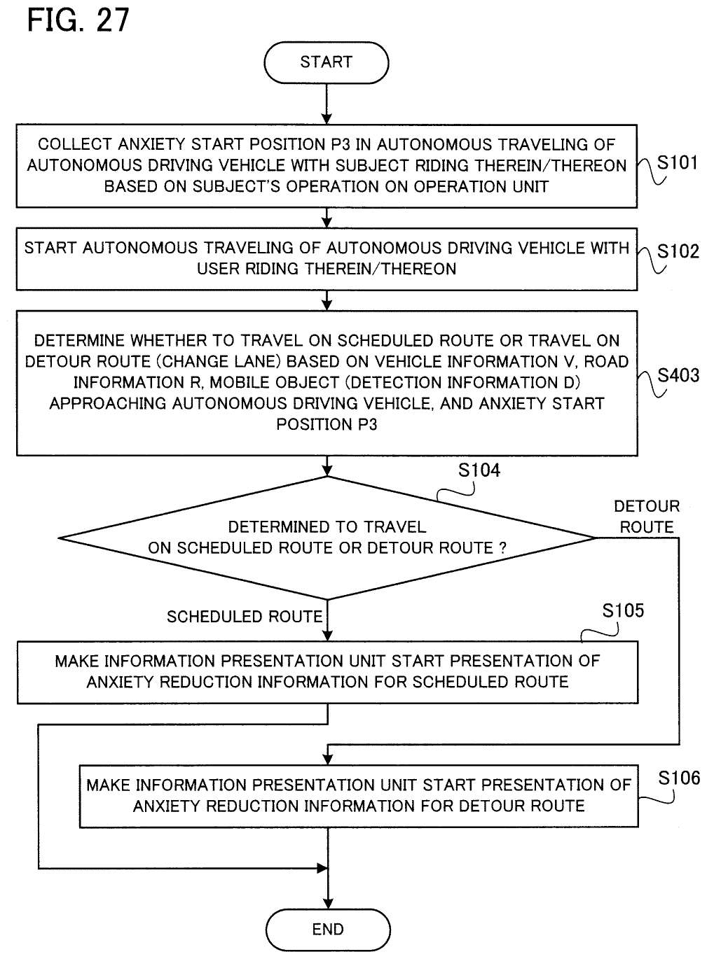

FIG. 27 is a flowchart showing the operation of the control device 10 of the autonomous driving vehicle 1 according to the fourth embodiment. In FIG. 27, each step being the same as a step shown in FIG. 14 is assigned the same reference character as in FIG. 14. The autonomous driving vehicle 1 according to the fourth embodiment differs from the autonomous driving vehicle 1 in the first embodiment in that the control unit 12 in step S403 regulates the autonomous traveling of the autonomous driving vehicle 1 based on one or more of the size and the speed of the mobile object (the vicinity detection information D) approaching the autonomous driving vehicle 1 and the distance to the mobile object and determines the detour route by changing the traveling lane to a different lane among a plurality of lanes (i.e., changing the traveling lane in the road width direction). Except for these features, the operation in FIG. 27 is the same as the operation in FIG. 14.

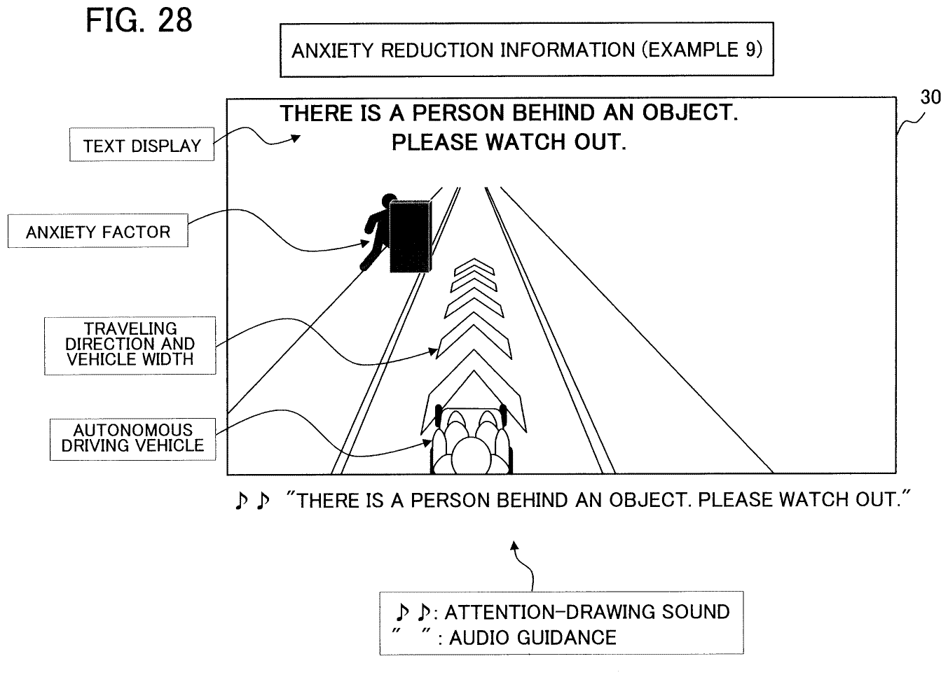

FIG. 28 is a diagram showing an example 9 of the anxiety reduction information presented by the information presentation unit 30 of the autonomous driving vehicle 1. FIG. 28 shows an example of the anxiety reduction information (an example of the display image, the attention-drawing sound and the audio guidance) in a state in which the anxiety factor 204 is a pedestrian as a mobile object and the pedestrian has started walking from behind an object on the roadside towards the center of the road. In this example, a display and audio guidance “THERE IS A PERSON BEHIND AN OBJECT. PLEASE WATCH OUT.” are shown. Also in this case, the control unit can execute the process shown in FIG. 27.

As described above, according to the fourth embodiment, the presentation of the anxiety reduction information is started based on the anxiety start position P3, and thus the presentation of the anxiety reduction information can be started even in a period in which the movement of the autonomous driving vehicle 1 does not change and the physiological index is hardly changed. Therefore, the anxiety reduction effect on the user 90 can be enhanced. Further, since the presentation start position P2 as the start position of the presentation of the anxiety reduction information is determined based on information previously collected based on reports from the experiment participants, the presentation of unnecessary information at positions where the presentation is unnecessary can be reduced.

Furthermore, it is also possible to incorporate the configuration and functions of the autonomous driving vehicle 1 according to the fourth embodiment into the autonomous driving vehicle according to any one of the first, second and third embodiments.

### Modifications

In the above-described first to fourth embodiments, the control unit 12 may make the information presentation unit 30 present the information by audio when no person in the vicinity of the autonomous driving vehicle 1 has been detected by the vicinity sensor 20 (e.g., when there is no person in a range specified by a predetermined distance) and make the information presentation unit 30 present the information by vibration when a person in the vicinity of the autonomous driving vehicle 1 has been detected by the vicinity sensor 20.

Further, in the above-described first to fourth embodiments, the user 90 of the autonomous driving vehicle in the autonomous traveling to the destination may report (e.g., input through the operation unit) the anxiety strength (i.e., the level of the feeling of anxiety) at each position where the user 90 felt anxiety. The control unit 12 may be provided with a learning function of recording position information and the anxiety strength in the storage device 50. In the next autonomous traveling and later, the control unit 12 may change the timing and the method of presenting the anxiety reduction information to the user 90 by referring to a model as the learned information.

Furthermore, the control device 10 may include processing circuitry to make an information presentation unit 30 for presenting information to the user present anxiety reduction information that reduces the anxiety felt by the user 90 when the autonomous driving vehicle 1 reaches a presentation start position P2 as a position before an anxiety start position P3 where the user 90 starts feeling anxiety about an anxiety factor 200 that makes the user feel anxiety in a scheduled route to a destination of the autonomous driving vehicle 1.

### DESCRIPTION OF REFERENCE CHARACTERS

1: autonomous driving vehicle, 10: control device, 11: collection unit, 12: control unit, 20: vicinity sensor, 21: forward camera, 22: laser radar, 30: information presentation unit (information presentation device), 31: display device, 32: speaker, 33: vibration device, 34: indicator lamp, 40: operation unit (operation device), 50: storage device, 51: GPS, 60: condition grasping sensor, 70: drive unit, 71: wheel, 72: steering, 73: seat, 74: speed sensor, 80: road, 80a: scheduled route, 80b: detour route, 80c: object, 90: user, 91: eye, 92: sight line, 101: processor, 102: memory, 103: nonvolatile storage device, 104: interface, 200-205: anxiety factor.

What is claimed is:

1. A control device provided in an autonomous driving vehicle that travels autonomously with a user riding therein, the control device comprising:
processing circuitry to make an information presentation device for presenting information to the user present anxiety reduction information that reduces the anxiety felt by the user when the autonomous driving vehicle reaches a presentation start position as a position before an anxiety start position where the user starts feeling anxiety about an anxiety factor that makes the user feel anxiety in a scheduled route to a destination of the autonomous driving vehicle.

2. An autonomous driving vehicle that travels autonomously with a user riding therein, comprising:
a vicinity sensor to detect at least a situation in front in a traveling direction;
an information presentation device to present information to the user;
an operation device; and
processing circuitry
to previously collect an anxiety start position, as a position before an anxiety factor that makes an experiment participant feel anxiety in a route of autonomous traveling of the autonomous driving vehicle with the experiment participant riding therein and a position where the experiment participant starts feeling anxiety about the anxiety factor, based on the experiment participant's operation on the operation device along the route; and
to control operation of the autonomous driving vehicle based on road information including a map of a scheduled route to a destination of the autonomous driving vehicle and a road attribute of the scheduled route, vehicle information regarding the autonomous driving vehicle, vicinity detection information outputted from the vicinity sensor, and the anxiety start position,
wherein the processing circuitry
sets a presentation start position, as a position where the information presentation device is made to start presenting anxiety reduction information, before the anxiety start position in the route of the autonomous traveling with the user riding therein, and
makes the information presentation device start the presentation of the anxiety reduction information when the autonomous driving vehicle with the user riding therein reaches the presentation start position.

3. The autonomous driving vehicle according to claim 2, wherein the processing circuitry sets the presentation start position between a recognition start position as a position where the experiment participant starts visually recognizing existence of the anxiety factor and the anxiety start position or at a same position as the recognition start position.

4. The autonomous driving vehicle according to claim 3, wherein the processing circuitry previously collects the recognition start position based on the experiment participant's operation on the operation device in the route of the autonomous traveling of the autonomous driving vehicle with the experiment participant riding therein along the route.

5. The autonomous driving vehicle according to claim 3, further comprising a condition grasping sensor to detect condition of the user riding in the autonomous driving vehicle,
wherein when it is judged that there exists a gaze target at which the user keeps on gazing for a time longer than or equal to a predetermined reference time based on the condition of the user detected by the condition grasping sensor, the processing circuitry handles a start position of the gazing as the recognition start position.

6. The autonomous driving vehicle according to claim 2, wherein the processing circuitry
makes a judgment on whether the autonomous driving vehicle is capable of passing through a position of the anxiety factor or not based on one or more items of information out of the road information, the vicinity detection information and the vehicle information,
determines whether making a detour around the position of the anxiety factor is necessary or not based on a result of the judgment,
makes the information presentation device present the anxiety reduction information that notifies the user that the detour will be made when the detour is made, and
makes the information presentation device present the anxiety reduction information that notifies the user that the autonomous driving vehicle travels without making the detour when the detour is not made.

7. The autonomous driving vehicle according to claim 2, wherein the processing circuitry
acquires a road width in the scheduled route as the anxiety factor from the road information or the vicinity detection information and acquires a vehicle width of the autonomous driving vehicle from the vehicle information,
makes a judgment on whether the autonomous driving vehicle is capable of passing through a position of the anxiety factor or not based on the road width and the vehicle width,
determines whether making a detour around the position of the anxiety factor is necessary or not based on a result of the judgment,
makes the information presentation device present the anxiety reduction information that notifies the user that the detour will be made when the detour is made, and
makes the information presentation device present the anxiety reduction information that notifies the user that the autonomous driving vehicle travels without making the detour when the detour is not made.

8. The autonomous driving vehicle according to claim 2, wherein the processing circuitry
acquires a road surface condition in the scheduled route as the anxiety factor from the road information or the vicinity detection information and acquires traveling performance of the autonomous driving vehicle from the vehicle information,
makes a judgment on whether the autonomous driving vehicle is capable of passing through a position of the anxiety factor or not based on the road surface condition and the traveling performance,
determines whether making a detour around the position of the anxiety factor is necessary or not based on a result of the judgment,
makes the information presentation device present the anxiety reduction information that notifies the user that the detour will be made when the detour is made, and
makes the information presentation device present the anxiety reduction information that notifies the user that the autonomous driving vehicle travels without making the detour when the detour is not made.

9. The autonomous driving vehicle according to claim 8, wherein the processing circuitry acquires one or more of an inclination of a slope in the scheduled route, a height of a step in the scheduled route, magnitude of unevenness in the scheduled route, and a condition of an unpaved surface in the scheduled route as the road surface condition.

10. The autonomous driving vehicle according to claim 2, wherein when the anxiety factor is judged to be a moving object based on the vicinity detection information from the vicinity sensor, the processing circuitry makes the information presentation device present a traveling position and a traveling speed of the autonomous driving vehicle as the anxiety reduction information.

11. The autonomous driving vehicle according to claim 10, wherein when the anxiety factor is judged to be an object moving in a direction of approaching the autonomous driving vehicle based on the vicinity detection information from the vicinity sensor, the processing circuitry makes the information presentation device present information regarding the object as the anxiety reduction information.

12. The autonomous driving vehicle according to claim 10, wherein the processing circuitry increases an enhancement level of a display mode of the information regarding the object with an increase in speed of the object, with the increase in size of the object, or with a decrease in distance to the object.

13. The autonomous driving vehicle according to claim 2, wherein the processing circuitry changes the scheduled route based on a user operation through the operation device.

14. The autonomous driving vehicle according to claim 2, wherein
the processing circuitry collects an anxiety strength level inputted by the user through the operation device in the autonomous traveling of the autonomous driving vehicle with the user as the experiment participant riding therein, and
the processing circuitry increases an enhancement level of a display mode of the information presented by the information presentation device with an increase in the anxiety strength level.

15. The autonomous driving vehicle according to claim 2, wherein the information presentation device includes at least one of a display device, a speaker, an indicator lamp and a vibration device.

16. The autonomous driving vehicle according to claim 2, wherein
the processing circuitry makes the information presentation device present the information by audio when no person in a vicinity of the autonomous driving vehicle has been detected by the vicinity sensor, and
the processing circuitry makes the information presentation device present the information by vibration when a person in the vicinity of the autonomous driving vehicle has been detected by the vicinity sensor.

17. The autonomous driving vehicle according to claim 2, wherein the autonomous driving vehicle is an autonomous driving wheelchair.

18. A control device of an autonomous driving vehicle that includes a vicinity sensor to detect at least a situation in front in a traveling direction, an information presentation device to present information to a user, and an operation device, the control device comprising:
processing circuitry
to previously collect an anxiety start position, as a position before an anxiety factor that makes an experiment participant feel anxiety in a route of autonomous traveling of the autonomous driving vehicle with the experiment participant riding therein and a position where the experiment participant starts feeling anxiety about the anxiety factor, based on the experiment participant's operation on the operation device along the route; and
to control operation of the autonomous driving vehicle based on road information including a map of a scheduled route to a destination of the autonomous driving vehicle and a road attribute of the scheduled route, vehicle information regarding the autonomous driving vehicle, vicinity detection information outputted from the vicinity sensor, and the anxiety start position,
wherein the processing circuitry
sets a presentation start position, as a position where the information presentation device is made to start presenting anxiety reduction information, before the anxiety start position in the route of the autonomous traveling with the user riding therein, and
makes the information presentation device start the presentation of the anxiety reduction information when the autonomous driving vehicle with the user riding therein reaches the presentation start position.

19. A control method of an autonomous driving vehicle that includes a vicinity sensor to detect at least a situation in front in a traveling direction, an information presentation device to present information to a user, and an operation device, the control method comprising:
previously collecting an anxiety start position, as a position before an anxiety factor that makes an experiment participant feel anxiety in a route of autonomous traveling of the autonomous driving vehicle with the experiment participant riding therein and a position where the experiment participant starts feeling anxiety about the anxiety factor, based on the experiment participant's operation on the operation device along the route; and
controlling operation of the autonomous driving vehicle based on road information including a map of a scheduled route to a destination of the autonomous driving vehicle and a road attribute of the scheduled route, vehicle information regarding the autonomous driving vehicle, vicinity detection information outputted from the vicinity sensor, and the anxiety start position,
wherein in the controlling the operation of the autonomous driving vehicle,
a presentation start position as a position where the information presentation device is made to start presenting anxiety reduction information is set before the anxiety start position in the route of the autonomous traveling with the user riding therein, and
the information presentation device is made to start the presentation of the anxiety reduction information when the autonomous driving vehicle with the user riding therein reaches the presentation start position.

20. A non-transitory computer-readable storage medium storing a control program of an autonomous driving vehicle to be executed by a control device of the autonomous driving vehicle that includes a vicinity sensor to detect at least a situation in front in a traveling direction, an information presentation device to present information to a user, and an operation device, wherein the control program causes the control device to execute:
previously collecting an anxiety start position, as a position before an anxiety factor that makes an experiment participant feel anxiety in a route of autonomous traveling of the autonomous driving vehicle with the experiment participant riding therein and a position where the experiment participant starts feeling anxiety about the anxiety factor, based on the experiment participant's operation on the operation device along the route; and
controlling operation of the autonomous driving vehicle based on road information including a map of a scheduled route to a destination of the autonomous driving vehicle and a road attribute of the scheduled route, vehicle information regarding the autonomous driving vehicle, vicinity detection information outputted from the vicinity sensor, and the anxiety start position, setting a presentation start position, as a position where the information presentation device is made to start presenting anxiety reduction information, before the anxiety start position in the route of the autonomous traveling with the user riding therein, and making the information presentation device start the presentation of the anxiety reduction information when the autonomous driving vehicle with the user riding therein reaches the presentation start position.
